(19) United States
(12) Patent Application Publication
(10) Pub. No.: US 2024/0316005 A1
(43) Pub. Date: Sep. 26, 2024

(54) GENE SIGNATURES FOR PREDICTING SURVIVAL TIME IN PATIENTS SUFFERING FROM RENAL CELL CARCINOMA

(71) Applicants: INSTITUT NATIONAL DE LA SANTÉ ET DE LA RECHERCHE MÉDICALE, Paris (FR); UNIVERSITÉ DE BORDEAUX, Bordeaux (FR); CENTRE NATIONAL DE LA RECHERCHE SCIENTIFIQUE (CNRS), Paris (FR); UNIVERSITÉ COTE D'AZUR, Nice (FR)

(72) Inventors: Andreas BIKFALVI, Pessac (FR); Wilfried SOULEYREAU, Pessac (FR); Lindsay COOLEY, Bordeaux (FR); Marie NIKOLSKI, Bordeaux Cedex (FR); Justine RUDEWICZ, Bordeaux cedex (FR); Gilles PAGES, Nice (FR); Maeva DUFIES, Nice (FR)

(21) Appl. No.: 18/575,431

(22) PCT Filed: Jul. 4, 2022
(86) PCT No.: PCT/EP2022/068483

§ 371 Date: Dec. 29, 2023

(30) Foreign Application Priority Data
Jul. 5, 2021 (EP) 21183633.3

(51) Int. Cl. A61K 31/404; A61K 39/395; A61P 35/00; C07K 16/28; C12Q 1/6886

(52) U.S. Cl. CPC A61K 31/404; A61K 39/3955; A61P 35/00; C07K 16/2818; C12Q 1/6886; C07K 2317/21; C12Q 2600/118; C12Q 2600/158

(57) ABSTRACT

Renal Cell Carcinoma (RCC) is difficult to treat with 5-year survival rate of 10% in metastatic patients. Main reasons of therapy failure are lack of validated biomarkers and scarce knowledge of the biological processes occurring during RCC progression. Thus, the investigation of mechanisms regulating RCC progression is fundamental to improve RCC therapy. In order to identify molecular markers and gene processes involved in the steps of RCC progression, the inventors generated several cell lines of higher aggressiveness by serially passaging mouse renal cancer RENCA cells in mice and, concomitantly, performed functional genomics analysis of the cells. Multiple cell lines depicting the major steps of tumor progression (including primary tumor growth, survival in the blood circulation and metastatic spread) were generated and analyzed by large-scale transcriptome, genome and methylome analyses. Importantly, transcriptome analysis revealed distinct signatures of tumor aggressiveness which were validated in the TCGA-KIRC cohort. The signatures are particularly suitable for determining the survival time of the patients and predicting response to the therapies.

### FIELD OF THE INVENTION

The present invention is in the field of medicine, in particular oncology.

### BACKGROUND OF THE INVENTION

Renal Cell Carcinoma (RCC) encompasses a heterogeneous group of cancers derived from renal tubular epithelial cells, including multiple histological and molecular sub-types, of which clear cell RCC (ccRCC) is the most common (1). RCC accounted for around 179,000 worldwide deaths during the last year, and its mortality is predicted to double in the next 20 years (Globocan project 2020, update December 2020 (2)). The major issue for RCC patients is the absence of an efficient therapeutic option, especially for recurrent and metastatic forms of the disease. Localized RCC is treated by surgical resection and has a good 5-year survival rate. However, 40% of patients with seemingly localized disease later relapse with localized or metastatic RCC. Both localized recurrence and RCC metastasis are difficult to treat and give a poor prognosis (3). The preferred therapeutic options for RCC treatment aim to target or tumor angiogenesis (i.e. Sunitinib or Bevacizumab, blockers of VEGF/VEGFR) (1) or the immune system (i.e. Ipilimumab+Nivolumab, two inhibitors of CTLA-4 and PD-1 respectively),. However, such therapies are rarely curative, and eventual drug resistance is almost inevitable. Furthermore, clinical treatment of RCC is hampered by a lack of relevant biomarkers. Patient diagnosis, prognosis and clinical decisions are currently based on histological information (i.e. Fuhrman grade and tumor stage (4,5), and therapy selection is based on limited guidelines and response to previous treatments. In this respect, clinical treatment of RCC lags behind other cancers for which molecular knowledge is invaluable in guiding clinical decisions (e.g. hormone receptor status in breast cancer). In fact, little is known about the pathophysiological mechanisms of RCC initiation and progression. The elucidation of such mechanisms is essential to undertake efficient therapeutic interventions for RCC, especially for its metastatic forms. Tumor progression from initiation to full metastasis is a multi-step process which occurs via a series of overlapping stages. Tumor progression can be seen as an evolutionary process whereby tumor cells must detach from the primary tumor, gain access to and survive in the circulation, exit the vasculature, and survive and proliferate in the environment of the secondary organ. Thus, different mechanisms come into play at different stages, and overall tumor progression is the sum of these processes (6). A better understanding of the molecular changes occurring in the cancer cell during tumor progression steps could aid in the diagnosis, prevention and treatment of metastatic cancer, including RCC.

WO2020182932 discloses several gene signatures that are suitable for predicting survival time in patients suffering from RCC.

### SUMMARY OF THE INVENTION

The present invention is defined by the claims. In particular, the present invention relates to new gene signatures that are suitable for predicting survival time in patients suffering from renal cell carcinoma (RCC).

### DETAILED DESCRIPTION OF THE INVENTION

Renal Cell Carcinoma (RCC) is difficult to treat with 5-year survival rate of 10% in metastatic patients. Main reasons of therapy failure are lack of validated biomarkers and scarce knowledge of the biological processes occurring during RCC progression. Thus, the investigation of mechanisms regulating RCC progression is fundamental to improve RCC therapy. In order to identify molecular markers and gene processes involved in the steps of RCC progression, the inventors generated several cell lines of higher aggressiveness by serially passaging mouse renal cancer RENCA cells in mice and, concomitantly, performed functional genomics analysis of the cells. Multiple cell lines depicting the major steps of tumor progression (including primary tumor growth, survival in the blood circulation and metastatic spread) were generated and analyzed by large-scale transcriptome, genome and methylome analyses. Through in vivo passaging, RENCA cells showed increased aggressiveness by reducing mice survival, enhancing primary tumor growth and lung metastases formation. In addition, transcriptome and methylome analyses showed distinct clustering of the cell lines. DNA sequencing did not show significant genomic variations in the different groups, indicating absence of clonal selection during the in vivo amplification process. Importantly, transcriptome analysis revealed distinct signatures of tumor aggressiveness which were validated in the TCGA-KIRC cohort. The signatures are particularly suitable for determining the survival time of the patients and predicting response to the therapies.

An object of the present invention relates to a method for predicting the survival time of a patient suffering from a renal cell carcinoma (RCC) comprising determining the expression levels of ADAM8, ARHGAP33, BTG3, COL6A1, CYBA, DNAJC12, DYRK4, FKBP10, IL34, MTMR7, PADI3, PLAU, RCN3, and TPRG1 (“KPT signature”).

A further object of the present invention relates to a method for predicting the survival time of a patient suffering from a renal cell carcinoma (RCC) comprising determining the expression levels of ARHGAP33, BTG3, C4orf48, CKAP4, CRABP2, CYP3A4, DNAJC12, DYRK4, EREG, GFPT2, HISTIHIE, KCTD17, KDELR3, MMP14, NCAMI, NME4, PIGZ, PLAU, PLOD2, RGS19, SERPINA3, TBX4, TMEM45A, and TPRG1 (“K-LM signature”).

A further object of the present invention relates to a method for predicting the survival time of a patient suffering from a renal cell carcinoma (RCC) comprising determining the expression levels of BCL3, CFB, COL6A1, CYP3A4, IQSEC3, KCTD17, KRT19, LOXL2, LRG1, PCBP3, SAA1, SAA2, SERPINA3, SOCS3, UCK2, and WT1 (“T-LM signature”).

As used herein, the term “renal cell carcinoma” or “RCC” has its general meaning in the art and refers to refers to a cancer originated from the renal tubular epithelial cells in the kidney. According to the pathological features, the cancer is classified into clear cell type, granular cell type, chromophobe type, spindle type, cyst-associated type, cyst-originating type, cystic type, or papillary type. In some embodiments, the renal cell carcinoma (RCC) is at Stage I, II, III, or IV as determined by the TNM classification, but however the present invention is accurately useful for predicting the survival time of patients when said cancer has been classified as Stage II or III by the TNM classification, i.e. non metastatic renal cell carcinoma (RCC).

The method of the present invention is particularly suitable for predicting the duration of the overall survival (OS), progression-free survival (PFS) and/or the disease-free survival (DFS) of the cancer patient. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they've become cancer-free (achieved remission). DSF gives more specific information and is the number of people with a particular cancer who achieve remission. Also, progression-free survival (PFS) rates (the number of people who still have cancer, but their disease does not progress) includes people who may have had some success with treatment, but the cancer has not disappeared completely. As used herein, the expression “short survival time” indicates that the patient will have a survival time that will be lower than the median (or mean) observed in the general population of patients suffering from said cancer. When the patient will have a short survival time, it is meant that the patient will have a “poor prognosis”. Inversely, the expression “long survival time” indicates that the patient will have a survival time that will be higher than the median (or mean) observed in the general population of patients suffering from said cancer. When the patient will have a long survival time, it is meant that the patient will have a “good prognosis”.

In some embodiments, the sample is a tissue tumor sample. The term “tumor tissue sample” means any tissue tumor sample derived from the patient. Said tissue sample is obtained for the purpose of the in vitro evaluation. In some embodiments, the tumor sample may result from the tumor resected from the patient. In some embodiments, the tumor sample may result from a biopsy performed in the primary tumour of the patient or performed in metastatic sample distant from the primary tumor of the patient. In some embodiments, the tumor tissue sample encompasses (i) a global primary tumor (as a whole), (ii) a tissue sample from the centre of the tumor, (iii) lymphoid islets in close proximity with the tumor, (iv) the lymph nodes located at the closest proximity of the tumor, (v) a tumor tissue sample collected prior surgery (for follow-up of patients after treatment for example), and (vi) a distant metastasis. In some embodiments, the tumor tissue sample, encompasses pieces or slices of tissue that have been removed from the tumor, including following a surgical tumor resection or following the collection of a tissue sample for biopsy, for further quantification of several expression levels of different genes, notably through histology or immunohistochemistry methods, through flow cytometry methods and through methods of gene or protein expression analysis, including genomic and proteomic analysis. The tumor tissue sample can, of course, be subjected to a variety of well-known post-collection preparative and storage techniques (e.g., fixation, storage, freezing, etc.). The sample can be fresh, frozen, fixed (e.g., formalin fixed), or embedded (e.g., paraffin embedded).

In the present specification, the name of each of the genes of interest (i.e. ADAM8, ARHGAP33, ARHGAP33, BCL3, C4orf48, CFB, CKAP4, COL6A1, CRABP2, CYBA, CYP3A4, DNAJC12, DYRK4, EREG, FKBP10, GFPT2, HISTIHIE, IL34, IQSEC3, KCTD17, KDELR3, KRT19, LOXL2, LRG1, MMP14, MTMR7, NCAMI, NME4, PADI3, PCBP3, PIGZ, PLAU, PLAU, PLOD2, RCN3, RGS19, SAA1, SAA2, SERPINA3, SOCS3, TBX4, TMEM45A, TPRG1, UCK2, and WT1) refers to the internationally recognized name of the corresponding gene, as found in internationally recognized gene sequences and protein sequences databases, including in the database from the HUGO Gene Nomenclature Committee that is available notably at the following Internet address: http://www.gene.ucl.ac.uk/nomenclature/index.html. In the present specification, the name of each of the genes of interest may also refer to the internationally recognized name of the corresponding gene, as found in the internationally recognized gene sequences and protein sequences database Genbank. Through these internationally recognized sequence databases, the nucleic acid and the amino acid sequences corresponding to each of the gene of interest described herein may be retrieved by the one skilled in the art.

As used herein, the term “expression level” refers, e.g., to a determined level of expression of gene of interest. The expression level of expression indicates the amount of expression product in a sample. The expression product of a gene of interest can be the nucleic acid of interest itself, a nucleic acid transcribed or derived therefrom, or a polypeptide or protein derived therefrom.

In some embodiments, the expression level of the gene is determined at nucleic acid level. Typically, the expression level of a gene may be determined by determining the quantity of mRNA. Methods for determining the quantity of mRNA are well known in the art. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e.g., Northern blot analysis, in situ hybridization) and/or amplification (e.g., RT-PCR). Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In some embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook-A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antl1ranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6dicl1lorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 nm (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6, 130, 101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can he detected as colored light of a specific wavelength or fluorescence. Semiconductor nanocrystals with different spectral characteristics are described in e.g., U.S. Pat. No. 6,602,671. Semiconductor nanocrystals that can be coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can be produced that are identifiable based on their different spectral characteristics. For example, semiconductor nanocrystals can be produced that emit light of different colors based on their composition, size or size and composition. For example, quantum dots that emit light at different wavelengths based on size (565 nm, 655 nm, 705 nm, or 800 nm emission wavelengths), which are suitable as fluorescent labels in the probes disclosed herein are available from Life Technologies (Carlshad, Calif.).

Additional labels include, for example, radioisotopes (such as 3 H), metal chelates such as DOTA and DPTA chelates of radioactive or paramagnetic metal ions like Gd3+, and liposomes. Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can be used in a metallographic detection scheme. For example, silver in situ hybridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. 1. Pathol. 157:1467-1472, 2000 and U.S. Pat. No. 6,942,970. Additional detection methods are provided in U.S. Pat. No. 6,280,929. Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will be appreciated by those of skill in the art that by appropriately selecting labelled probe-specific binding agent pairs, multiplex detection schemes can he produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can he labelled with a first hapten, such as biotin, while a second probe that corresponds to a second target sequence can be labelled with a second hapten, such as DNP. Following exposure of the sample to the probes, the bound probes can he detected by contacting the sample with a first specific binding agent (in this case avidin labelled with a first fluorophore, for example, a first spectrally distinct QUANTUM DOT®, e.g., that emits at 585 nm) and a second specific binding agent (in this case an anti-DNP antibody, or antibody fragment, labelled with a second fluorophore (for example, a second spectrally distinct QUANTUM DOT®, e.g., that emits at 705 nm). Additional probes/binding agent pairs can he added to the multiplex detection scheme using other spectrally distinct fluorophores. Numerous variations of direct, and indirect (one step, two step or more) can he envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5x or 6x SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In some embodiments, the methods of the invention comprise the steps of providing total RNAs extracted from the sample and subjecting the RNAs to amplification and hybridization to specific probes, more particularly by means of a quantitative or semi-quantitative RT-PCR.

In some embodiments, the level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In some embodiments, the nCounter® Analysis system is used to detect intrinsic gene expression. The basis of the nCounter® Analysis system is the unique code assigned to each nucleic acid target to be assayed (International Patent Application Publication No. WO 08/124847, U.S. Pat. No. 8,415,102 and Geiss et al. Nature Biotechnology. 2008. 26(3): 317-325; the contents of which are each incorporated herein by reference in their entireties). The code is composed of an ordered series of colored fluorescent spots which create a unique barcode for each target to be assayed. A pair of probes is designed for each DNA or RNA target, a biotinylated capture probe and a reporter probe carrying the fluorescent barcode. This system is also referred to, herein, as the nanoreporter code system. Specific reporter and capture probes are synthesized for each target. The reporter probe can comprise at a least a first label attachment region to which are attached one or more label monomers that emit light constituting a first signal; at least a second label attachment region, which is non-over-lapping with the first label attachment region, to which are attached one or more label monomers that emit light constituting a second signal; and a first target-specific sequence. Preferably, each sequence specific reporter probe comprises a target specific sequence capable of hybridizing to no more than one gene and optionally comprises at least three, or at least four label attachment regions, said attachment regions comprising one or more label monomers that emit light, constituting at least a third signal, or at least a fourth signal, respectively. The capture probe can comprise a second target-specific sequence; and a first affinity tag. In some embodiments, the capture probe can also comprise one or more label attachment regions. Preferably, the first target-specific sequence of the reporter probe and the second target-specific sequence of the capture probe hybridize to different regions of the same gene to be detected. Reporter and capture probes are all pooled into a single hybridization mixture, the “probe library”. The relative abundance of each target is measured in a single multiplexed hybridization reaction. The method comprises contacting the tissue sample with a probe library, such that the presence of the target in the sample creates a probe pair-target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies). Purified reactions are typically deposited by the Prep Station into individual flow cells of a sample cartridge, bound to a streptavidin-coated surface via the capture probe, electrophoresed to elongate the reporter probes, and immobilized. After processing, the sample cartridge is transferred to a fully automated imaging and data collection device (Digital Analyzer, NanoString Technologies). The expression level of a target is measured by imaging each sample and counting the number of times the code for that target is detected. For each sample, typically 600 fields-of-view (FOV) are imaged (1376×10²⁴ pixels) representing approximately 10 mm2 of the binding surface. Typical imaging density is 100-1200 counted reporters per field of view depending on the degree of multiplexing, the amount of sample input, and overall target abundance. Data is output in simple spreadsheet format listing the number of counts per target, per sample. This system can be used along with nanoreporters. Additional disclosure regarding nanoreporters can be found in International Publication No. WO 07/076129 and WO07/076132, and US Patent Publication No. 2010/0015607 and 2010/0261026, the contents of which are incorporated herein in their entireties. Further, the term nucleic acid probes and nanoreporters can include the rationally designed (e.g. synthetic sequences) described in International Publication No. WO 2010/019826 and US Patent Publication No. 2010/0047924, incorporated herein by reference in its entirety.

Level of a gene may be expressed as absolute level or normalized level. Typically, levels are normalized by correcting the absolute level of a gene by comparing its expression to the expression of a gene that is not a relevant for determining the risk. This normalization allows the comparison of the level in one sample, e.g., a subject sample, to another sample, or between samples from different sources.

In some embodiments, a score which is a composite of the expression levels of the different genes is determined and compared to a predetermined reference value wherein a difference between said score and said predetermined reference value is indicative whether the subject will have a long or short survival time.

The score can be calculated in any appropriate manner, such as principal components analysis, support vector machines, or other techniques known to the person of ordinary skill in the art having the benefit of the present disclosure.

In some embodiments, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of the score in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the score in a group of reference, one can use algorithmic analysis for the statistic treatment of the measured expression levels of the gene(s) in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc. In some embodiments, the predetermined reference value is determined by carrying out a method comprising the steps of a) providing a collection of samples; b) providing, for each sample provided at step a), information relating to the actual clinical outcome for the corresponding subject (i.e. the duration of the survival); c) providing a serial of arbitrary quantification values; d) determining the expression levels of different genes for each sample contained in the collection provided at step a) so as to calculate the score as described above; e) classifying said samples in two groups for one specific arbitrary quantification value provided at step c), respectively: (i) a first group comprising samples that exhibit a quantification value for the score that is lower than the said arbitrary quantification value contained in the said serial of quantification values; (ii) a second group comprising samples that exhibit a quantification value for said score that is higher than the said arbitrary quantification value contained in the said serial of quantification values; whereby two groups of samples are obtained for the said specific quantification value, wherein the samples of each group are separately enumerated; f) calculating the statistical significance between (i) the quantification value obtained at step e) and (ii) the actual clinical outcome of the patients from which samples contained in the first and second groups defined at step f) derive; g) reiterating steps f) and g) until every arbitrary quantification value provided at step d) is tested; h) setting the said predetermined reference value as consisting of the arbitrary quantification value for which the highest statistical significance (most significant) has been calculated at step g).

For example the score has been assessed for 100 samples of 100 patients. The 100 samples are ranked according to the determined score. Sample 1 has the highest score and sample 100 has the lowest score. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding subject, Kaplan Meier curves are prepared for each of the 99 groups of two subsets. Also for each of the 99 groups, the p value between both subsets was calculated. The predetermined reference value is then selected such as the discrimination based on the criterion of the minimum p value is the strongest. In other terms, the score corresponding to the boundary between both subsets for which the p value is minimum is considered as the predetermined reference value.

Typically, a score that is higher than the predetermined reference value indicates that the patient will have a short survival time and a score that is lower than the predetermined reference value indicates that the patient will have a long survival time.

In some embodiments, the predetermined reference value thus allows discrimination between a poor and a good prognosis for a patient. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above. For example, according to this specific embodiment of a “cut-off” value, the outcome can be determined by comparing the calculated score with the range of values which are identified. In some embodiments, a cut-off value thus consists of a range of quantification values, e.g. centered on the quantification value for which the highest statistical significance value is found (e.g. generally the minimum p value which is found). For example, on a hypothetical scale of 1 to 10, if the ideal cut-off value (the value with the highest statistical significance) is 5, a suitable (exemplary) range may be from 4-6. For example, a patient may be assessed by comparing values obtained by measuring the calculated score, where values higher than 5 reveal a poor prognosis and values less than 5 reveal a good prognosis. In some embodiments, a patient may be assessed by comparing values obtained by measuring the calculated score and comparing the values on a scale, where values above the range of 4-6 indicate a poor prognosis and values below the range of 4-6 indicate a good prognosis, with values falling within the range of 4-6 indicating an intermediate occurrence (or prognosis).

Typically, overexpression of the genes of each signature of the present invention correlates with a poor prognosis.

In some embodiments, the method of the invention comprises the use of a classification algorithm typically selected from Linear Discriminant Analysis (LDA), Topological Data Analysis (TDA), Neural Networks, Support Vector Machine (SVM) algorithm and Random Forests algorithm (RF) such as described in the Example. In some embodiments, the method of the invention comprises the step of determining the patient's survival using a classification algorithm.

As used herein, the term “classification algorithm” has its general meaning in the art and refers to classification and regression tree methods and multivariate classification well known in the art such as described in U.S. Pat. No. 8,126,690; WO2008/156617. As used herein, the term “support vector machine (SVM)” is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase. In general, SVMs provide a model for use in classifying each of n subjects to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction (Vapnik, 1998 Statistical Learning Theory. New York: Wiley). Any new observation is then classified as belonging to any one of the categories of interest, based where the observation lies in relation to the hyperplane. When more than two categories are considered, the process is carried out pairwise for all of the categories and those results combined to create a rule to discriminate between all the categories. As used herein, the term “Random Forests algorithm” or “RF” has its general meaning in the art and refers to classification algorithm such as described in U.S. Pat. No. 8,126,690; WO2008/156617. Random Forest is a decision-tree-based classifier that is constructed using an algorithm originally developed by Leo Breiman (Breiman L, “Random forests,” Machine Learning 2001, 45:5-32). The classifier uses a large number of individual decision trees and decides the class by choosing the mode of the classes as determined by the individual trees. The individual trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set. In some embodiments, the score is generated by a computer program.

In some embodiments, the method of the present invention comprises a) quantifying the level of a plurality of genes in the sample; b) implementing a classification algorithm on data comprising the quantified plurality of genes so as to obtain an algorithm output; c) determining the survival time from the algorithm output of step b).

The algorithm of the present invention can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

In some embodiment, in view of the currently limited options for RCC management, the group of biomarkers as disclosed herein is useful for identifying patients with poor-prognosis, in particular patients with localized RCCs that are likely to relapse and metastasize. Accordingly, subject identified with a poor prognosis can be administered therapy, for example systematic therapy. In some embodiments, the method of the present invention be used to identify patients in need of frequent follow-up by a physician or clinician to monitor RCC disease progression. Screening patients for identifying patients having a poor prognosis as disclosed herein is also useful to identify patients most suitable or amenable to be enrolled in clinical trial for assessing a therapy for RCC, which will permit more effective subgroup analyses and follow-up studies. Furthermore, the expression levels as disclosed herein can be monitored in patients enrolled in a clinical trial to provide a quantitative measure for the therapeutic efficacy of the therapy which is subject to the clinical trial.

This invention also provides a method for selecting a therapeutic regimen or determining if a certain therapeutic regimen is more appropriate for a patient identified as having a poor prognosis as identified by the methods as disclosed herein. For example, an aggressive anti-cancer therapeutic regime can be perused in which a patient having a poor prognosis, where the patient is administered a therapeutically effective amount of an anti-cancer agent to treat the RCC. In some embodiments, a patient can be monitored for RCC using the methods as disclosed herein, and if on a first (i.e. initial) testing the patient is identified as having a poor prognosis, the patient can be administered an anti-cancer therapy, and on a second (i.e. follow-up testing), the patient is identified as having a good prognosis, the patient can be administered an anti-cancer therapy at a maintenance dose. In some embodiments, a patient can be monitored for RCC using the methods as disclosed herein, and if on a first (i.e. initial) testing the patient is identified as having a poor prognosis, the patient is administered an anti-cancer therapy, and on a second (i.e. follow-up) testing, the patient is identified as having a good prognosis, the patient is administered an anti-cancer therapy at a maintenance dose. In some embodiments, the present invention relates to a method for monitoring RCC in a patient comprising administering to said patient an anti-cancer therapy when the patient is identified as having a poor prognosis on a first (i.e. initial) testing and/or comprising administering an anti-cancer therapy at a maintenance dose when on a second (i.e. follow-up) testing the patient is identified as having a good prognosis.

The method of the present invention is particularly suited to determining which patients will be responsive or experience a positive treatment outcome to a treatment.

In general, a therapy is considered to “treat” RCC if it provides one or more of the following treatment outcomes: reduce or delay recurrence of the RCC after the initial therapy; increase median survival time or decrease metastases. In some embodiments, an anti-cancer therapy is, for example but not limited to administration of a chemotherapeutic agent, radiotherapy etc. Such anti-cancer therapies are disclosed herein, as well as others that are well known by persons of ordinary skill in the art and are encompassed for use in the present invention. The term “anti-cancer agent” or “anti-cancer drug” is any agent, compound or entity that would be capably of negatively affecting the cancer in the patient, for example killing cancer cells, inducing apoptosis in cancer cells, reducing the growth rate of cancer cells, reducing the number of metastatic cells, reducing tumor size, inhibiting tumor growth, reducing blood supply to a tumor or cancer cells, promoting an immune response against cancer cells or a tumor, preventing or inhibiting the progression of cancer, or increasing the lifespan of the patient with cancer. Anti-cancer therapy includes biological agents (biotherapy), chemotherapy agents, and radiotherapy agents. In some embodiments, the anti-cancer therapy includes a chemotherapeutic regimen further comprises radiation therapy. In some embodiments, the anti-cancer treatment comprises chemotherapy, alone or in combination with immunotherapy of the tumor. In some embodiments, the treatment comprises both chemotherapy and immunotherapy.

The term “chemotherapeutic agent” or “chemotherapy agent” are used interchangeably herein and refers to an agent that can be used in the treatment of RCC. In some embodiments, a chemotherapeutic agent can be in the form of a prodrug which can be activated to a cytotoxic form. Chemotherapeutic agents are commonly known by persons of ordinary skill in the art and are encompassed for use in the present invention. For example, chemotherapeutic drugs include, but are not limited to: temozolomide (Temodar), procarbazine (Matulane), and lomustine (CCNU). Chemotherapy given intravenously (by IV, via needle inserted into a vein) includes vincristine (Oncovin or Vincasar PFS), cisplatin (Platinol), carmustine (BCNU, BICNU), and carboplatin (Paraplatin), Mexotrexate (Rheumatrex or Trexall), irinotecan (CPT-11); erlotinib; oxalipatin; anthracyclins-idarubicin and daunorubicin; doxorubicin; alkylating agents such as melphalan and chlorambucil; cis-platinum, methotrexate, and alkaloids such as vindesine and vinblastine.

In some embodiments, the chemotherapy comprises administering anti-VEGF agents. As used herein the term “anti-VEGF agent” refers to any compound or agent that produces a direct effect on the signaling pathways that promote growth, proliferation and survival of a cell by inhibiting the function of the VEGF protein, including inhibiting the function of VEGF receptor proteins. The term “agent” or “compound” as used herein means any organic or inorganic molecule, including modified and unmodified nucleic acids such as antisense nucleic acids, RNAi agents such as siRNA or shRNA, peptides, peptidomimetics, receptors, ligands, and antibodies. Preferred VEGF inhibitors, include for example, AVASTIN® (bevacizumab), an anti-VEGF monoclonal antibody of Genentech, Inc. of South San Francisco, Calif., VEGF Trap (Regeneron/Aventis). Additional VEGF inhibitors include CP-547,632 (3-(4-Bromo-2,6-difluoro-benzyloxy)-5-[3-(4-pyrrolidin 1-yl-butyl)-ureido]-isothiazole-4-carboxylic acid amide hydrochloride; Pfizer Inc., NY), AG13736, AG28262 (Pfizer Inc.), SU5416, SU11248, & SU6668 (formerly Sugen Inc., now Pfizer, New York, N.Y.), ZD-6474 (AstraZeneca), ZD4190 which inhibits VEGF-R2 and -R1 (AstraZeneca), CEP-7055 (Cephalon Inc., Frazer, Pa.), PKC 412 (Novartis), AEE788 (Novartis), AZD-2171), NEXAVAR® (BAY 43-9006, sorafenib; Bayer Pharmaceuticals and Onyx Pharmaceuticals), vatalanib (also known as PTK-787, ZK-222584: Novartis & Schering: AG), MACUGEN® (pegaptanib octasodium, NX-1838, EYE-001, Pfizer Inc./Gilead/Eyetech), IM862 (glufanide disodium, Cytran Inc. of Kirkland, Wash., USA), VEGFR2-selective monoclonal antibody DC101 (ImClone Systems, Inc.), angiozyme, a synthetic ribozyme from Ribozyme (Boulder, Colo.) and Chiron (Emeryville, Calif.), Sirna-027 (an siRNA-based VEGFR1 inhibitor, Sirna Therapeutics, San Francisco, Calif.) Caplostatin, soluble ectodomains of the VEGF receptors, Neovastat (AEterna Zentaris Inc; Quebec City, Calif.) and combinations thereof. In some embodiments, the anti-VEGF agent is sunitinib (N-[2-(diethylamino)ethyl]-5-{[(3Z)-5-fluoro-2-oxo-2,3-dihydro-1H-indol-3-ylidene]methyl}-2,4-dimethyl-1H-pyrrole-3-carboxamide).

As used herein, the term “immunotherapy” has its general meaning in the art and refers to the treatment that consists in administering an immunogenic agent i.e. an agent capable of inducing, enhancing, suppressing or otherwise modifying an immune response.

In some embodiments, the immunotherapy consists in administering the patient with at least one immune checkpoint inhibitor.

As used herein, the term “immune checkpoint inhibitor” has its general meaning in the art and refers to any compound inhibiting the function of an immune inhibitory checkpoint protein. As used herein the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of inhibitory checkpoint molecules include A2AR, B7-H3, B7-H4, BTLA, CTLA-4, CD277, IDO, KIR, PD-1, LAG-3, TIM-3 and VISTA. Inhibition includes reduction of function and full blockade. Preferred immune checkpoint inhibitors are antibodies that specifically recognize immune checkpoint proteins. A number of immune checkpoint inhibitors are known and in analogy of these known immune checkpoint protein inhibitors, alternative immune checkpoint inhibitors may be developed in the (near) future. The immune checkpoint inhibitors include peptides, antibodies, nucleic acid molecules and small molecules. Examples of immune checkpoint inhibitor includes PD-1 antagonist, PD-L1 antagonist, PD-L2 antagonist CTLA-4 antagonist, VISTA antagonist, TIM-3 antagonist, LAG-3 antagonist, IDO antagonist, KIR2D antagonist, A2AR antagonist, B7-H3 antagonist, B7-H4 antagonist, and BTLA antagonist.

In some embodiments, PD-1 (Programmed Death-1) axis antagonists include PD-1 antagonist (for example anti-PD-1 antibody), PD-L1 (Programmed Death Ligand-1) antagonist (for example anti-PD-L1 antibody) and PD-L2 (Programmed Death Ligand-2) antagonist (for example anti-PD-L2 antibody). In some embodiments, the anti-PD-1 antibody is selected from the group consisting of MDX-1106 (also known as Nivolumab, MDX-1106-04, ONO-4538, BMS-936558, and Opdivo®), Merck 3475 (also known as Pembrolizumab, MK-3475, Lambrolizumab, Keytruda®, and SCH-900475), and CT-011 (also known as Pidilizumab, hBAT, and hBAT-1). In some embodiments, the PD-1 binding antagonist is AMP-224 (also known as B7-DCIg). In some embodiments, the anti-PD-L1 antibody is selected from the group consisting of YW243.55.S70, MPDL3280A, MDX-1105, and MEDI4736. MDX-1105, also known as BMS-936559, is an anti-PD-L1 antibody described in WO2007/005874. Antibody YW243.55. S70 is an anti-PD-L1 described in WO 2010/077634 A1. MEDI4736 is an anti-PD-L1 antibody described in WO2011/066389 and US2013/034559. MDX-1106, also known as MDX-1106-04, ONO-4538 or BMS-936558, is an anti-PD-1 antibody described in U.S. Pat. No. 8,008,449 and WO2006/121168. Merck 3745, also known as MK-3475 or SCH-900475, is an anti-PD-1 antibody described in U.S. Pat. No. 8,345,509 and WO2009/114335. CT-011 (Pidizilumab), also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342. Atezolimumab is an anti-PD-L1 antibody described in U.S. Pat. No. 8,217,149. Avelumab is an anti-PD-L1 antibody described in U.S. Pat. No. 20,140,341917. CA-170 is a PD-1 antagonist described in WO2015033301 & WO2015033299. Other anti-PD-1 antibodies are disclosed in U.S. Pat. Nos. 8,609,089, 20,100,28330, and/or U.S. Pat. No. 20,120,114649. In some embodiments, the PD-1 inhibitor is an anti-PD-1 antibody chosen from Nivolumab, Pembrolizumab or Pidilizumab. In some embodiments, PD-L1 antagonist is selected from the group comprising of Avelumab, BMS-936559, CA-170, Durvalumab, MCLA-145, SP142, STI-A1011, STIA1012, STI-A1010, STI-A1014, A110, KY1003 and Atezolimumab and the preferred one is Avelumab, Durvalumab or Atezolimumab.

In some embodiments, CTLA-4 (Cytotoxic T-Lymphocyte Antigen-4) antagonists are selected from the group consisting of anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (Ipilimumab), Tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, inhibitors of CTLA-4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B. Additional CTLA-4 antibodies are described in U.S. Pat. Nos. 5,811,097; 5,855,887; 6,051,227; and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin: Oncology, 22(145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281. A preferred clinical CTLA-4 antibody is human monoclonal antibody (also referred to as MDX-010 and Ipilimumab with CAS No. 477202-00-9 and available from Medarex, Inc., Bloomsbury, N.J.) is disclosed in WO 01/14424. With regard to CTLA-4 antagonist (antibodies), these are known and include Tremelimumab (CP-675,206) and Ipilimumab.

In some embodiments, the immunotherapy consists in administering to the patient a combination of a CTLA-4 antagonist (e.g. Ipilimumab) and a PD-1 antagonist (e.g. Nivolumab or Pembrolizumab).

The compounds used in connection with the treatment methods of the present invention are administered and dosed in accordance with good medical practice, taking into account the clinical condition of the individual subject, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including, but not limited to, improved survival rate or more rapid recovery, or improvement or elimination of symptoms and other indicators as are selected as appropriate measures by those skilled in the art.

The invention will be further illustrated by the following figures and examples. However, these examples and figures should not be interpreted in any way as limiting the scope of the present invention.

### FIGURES

FIG. 1. Strategy used for RCC biomarkers discovery and validation. The Figure depicts the general strategy used to determine clinically relevant signatures from each generated cell line (i.e. KPT, K-LM and T-LM). For detailed description see Materials and Methods section.

FIG. 2. Validation of the KPT signature in the KIRC-TCGA cohort. a and b Kaplan-Meier for Overall Survival (OS) and Disease-Free Survival (DFS) analysis stratified in 3 groups of equivalent size based on the signature score. “low”: patient group with low score; “medium”: patient group with a medium score; “high”: patient group with high score. c and d Density plot of p values (log-rank test) from 1000 random signatures of equal size for OS and DFS. p=empirical p-value. e and f Forest plot of multivariate Cox proportional hazards model for the KPT signature in the KIRC cohort adjusted for clinical variables (Fuhrman grade and TNM stage).

FIG. 3. Validation of the T-LM signature in the KIRC-TCGA cohort. a and b Kaplan-Meier for Overall Survival (OS) and Disease-Free Survival (DFS) analysis stratified in 3 groups of equivalent size based on the signature score. “low”: patient group with low score; “medium”: patient group with a medium score; “high”: patient group with high score. c and d Density plot of p values (log-rank test) from 1000 random signatures of equal size for OS and DFS. p=empirical p-value. e and f Forest plot of multivariate Cox proportional hazards model for the T-LM signature in the KIRC cohort adjusted for clinical variables (Fuhrman grade and TNM stage).

FIG. 4. Validation of the K-LM signature in the KIRC-TCGA cohort. a and b Kaplan-Meier for Overall Survival (OS) and Disease-Free Survival (DFS) analysis stratified in 3 groups of equivalent size based on the signature score. “low”: patient group with low score; “medium”: patient group with a medium score; “high”: patient group with high score. c and d Density plot of p values (log-rank test) from 1000 random signatures of equal size for OS and DFS. p=empirical p-value. e and f Forest plot of multivariate Cox proportional hazards model for the Lung K-LM signature in the KIRC cohort adjusted for clinical variables (Fuhrman grade and TNM stage).

FIG. 5. Signatures in the Nivolumab cohort. The KPT (a), T-LM (b), K-LM (c) signatures are analyzed in the patient cohort treated with Nivolumab (left panels from top to bottom) for OS. As control, 1000 randomly-selected signatures are also tested (right panels, from top to bottom).

### EXAMPLE

### Methods

Cell culture

The murine renal cancer RENCA cell lines were maintained in RPMI-1640 (Eurobio) supplemented with 10%(v/v) FBS and 1%(v/v) penicillin-streptomycin, and incubated at 37°° C. with 5% CO₂. For the generation of GFP-expressing cells, a lentiviral vector (pRRLsin-MND-eGFP-WPRE) was obtained from the vectorology platform of the University of Bordeaux (Vect′UB), and used for infection of RENCA cells. Authentication of parental cell line was conducted by Microsynth on December 2020.

### GFP-RENCA in Mice Implantation-Extraction and Purification

For sub-capsular implantations, 1×10⁵ GFP-RENCA cells were injected under the left kidney capsule of 6 weeks old female BALB/c mice (Charles River Laboratories), whilst for intravenous injections 5×10⁶ cells were injected into the caudal vein. When a mouse from a group reached an endpoint, all mice from that group were sacrificed, and both primary tumors or lung metastases were collected. For tumor cell extraction and purification, tissues were cut into small pieces with a scalpel and digested with Collagenase I and Collagenase II (Liberase TL, Roche) for 1 hour at 37° C. Subsequently, digested tissues were filtered using cell strainers (100 μm, 70 μm and 40 μm) and cultured in complete RPMI-1640. Cell cultures were checked daily and passaged as necessary for around 2 weeks, until GFP-negative cells could not be detected. GFP-RENCA purity was assessed either by fluorescence microscopy or flow cytometry using BD Accuri C6 (BD Bioscience). Finally, GFP-RENCA cell lines were collected for analysis or re-implanted into a new set of mice for the subsequent in-vivo passage. Mice were housed in the animal facility of Bordeaux University (Animalerie Mutualisée, Université de Bordeaux, France). All animal experiments were approved by the “Ministère de l′Enseignement Supérieur, de la Recherche et de l'Innovation (MESRI)” (authorization numbers 2016072015478042; 2015110618597936 and 2015070315335217), and were carried out in accordance with the approved protocols.

### Transwell Migration Assay

The migratory properties of tumor cells were analyzed in Transwell chamber without membrane coating (24-well insert, 8 μm pore size, cat 353097, Falcon). 5×10⁵ GFP-RENCA cells/insert (P0, P1 or P6) were seeded in the top chamber using serum-free medium, while the lower chamber contained complete medium with 10% FBS. After 24 hours, cells were fixed in 4% PFA (sc-281692, Santa Cruz Biotechnology) and stained with DAPI. Non-migrated cells on the top of the insert membrane were removed using a cotton swap. Number of migrated cells was analyzed by fluorescent microscopy and counted as cells per acquired field using Fiji software.

RNA Extraction, Transcriptomic and qPCR Gene Expression Analyses

Total RNA was extracted using the RNeasy Plus Mini Kit (#74134, Qiagen), according to the manufacturer's protocols. For the analysis of the transcriptomic profiles of generated cancer cell lines, we used SurePrint G3 Mouse Gene Expression Microarrays (G4852A, Agilent). Instead, for Real-Time qPCR analysis, lug of total RNA was reverse-transcribed using the high-capacity cDNA reverse transcription kit (Applied Biosystems, 4368814). Then, cDNAs were analyzed using either EurobioProbe or EurobioGreen master mix (Eurobio Scientific), and StepOne Real-Time PCR System (Applied Biosystems). Human HPRTI or mouse a-Tubulin were used as housekeeping genes.

### Tissues Processing and Immunofluorescent Analysis

For GFP-RENCA purity assessment after extraction from animal tissues, cells were fixed with 4% PFA and stained with both DAPI and Alexa-Fluor 546 Phalloidin (A22283, Invitrogen) for visualization of GFP-positive and GFP-negative cells. For histological analysis, whole murine organs were fixed in 4% PFA (sc-281692, Santa Cruz Biotechnology) for 2 hours and then incubated for 72 hours in 30% sucrose at 4° C. Then, samples were embedded with OCT Compound (4583, Tissue-Tek OCT compound, Sakura). Before embedding, lungs were inflated with 1 mL of diluted OCT (1:1 PBS/OCT dilution). Finally, frozen organs were stored at −80° C. For staining of proliferative cancer cells in primary tumors, 10 μm sections were prepared using a cryostat (Leica CM1900) and furtherly fixed with 4% PFA for 15 minutes. Then, tissues were boiled for 10 minutes in sodium citrate buffer (10 mM, pH6) using a microwave, and blocked with 3% BSA-PBS. Anti-Ki67 primary antibody (1:250, ab16667, Abcam) and FluoProbes® 547H Donkey Anti-Rabbit secondary antibody (FP-SB5110, Interchim) were used at RT for 2 hours or 30 minutes, respectively. Finally, cell nuclei were counterstained with DAPI. Fluorescent microscopy was performed with a Nikon Eclipse i90 microscope (Nikon) and NIS-Elements AR 4.30 software (Nikon). A slide scanner (Hamamatsu, Nanozoomer 2.0HT) from the Bordeaux Imaging Center was used for whole slide imaging using NDP.scan software (Hamamatsu). Analysis were performed using Fiji.

### ELISA

Human plasma samples were collected from UroCCR cohort and analyzed by ELISA, according to the manufacturer's protocols: human SAA2 (DLdevelop, DL-SAA2-Hu-96T), Human CFB (Abcam, ab137973).

### Low-Coverage Whole-Genome Sequencing

Genomic DNA was isolated use the Dneasy Blood and Tissue Kit (cat 69504, Qiagen), according to the manufacturer's protocols. Libraries were created using the KAPA LTP library preparation kit (Kapa Biosystems) following the manufacturer's recommendations, and sequenced on an Illumina HiSeq4000 sequencer in 51 bp single-end mode. Raw reads were mapped to the mouse reference genome (mm 10/GRCm38) using the Burrows-Wheeler Aligner (35). On average, 6,882,693 reads were mapped per sample. PCR duplicates were removed using Picard (v1.43) resulting in, on average, 5,966,979 uniquely mapped reads per sample. These reads were further processed with the Bioconductor package QDNASeq.mm10. Problematic regions were excluded and read counts were corrected for mappability using LOESS regression. The number of reads was counted in bins of 50 kb along the genome, log 2-transformed and normalized by the median. These resulted in logR values per bin, that were subsequently segmented using the ASCAT algorithm (36). These segmented values, as well as the individual logR values per bin, were used to plot the individual copy-number profile of each sample. Subclonal tumor fractions of the samples were estimated by ABSOLUTE (37).

### Transcriptome Data Generation and Analysis

Counts of samples by group and passages:

| Group | P0 | P2 | P3 | P4 | P5 | P6 |
|---|---|---|---|---|---|---|
| Parental cell line | 2 | NA | NA | NA | NA | NA |
| KPT | NA | 2 | 2 | 3 | 5 | 5 |
| T-LM | NA | 2 | 3 | 4 | 6 | 4 |
| L-LM | NA | 1 | 5 | 3 | 4 | 3 |

From the log 2 scale normalized data set, Principal Component Analysis (PCA, function prcomp of stats R package (v3.6.2) with the parameter center=T) was performed on parental cell line (P0) and each passage resulting in P0-P3, P0-P5 and P0-P6 PCA. For the P0-P6 PCA, genes with the most important association were selected by keeping genes whose contributions were above the mean of all contributions for PC1 and PC2. This resulted in a set of 5194 genes. The mean value was computed for each passage P0 to P6 and used for the heatmap using the pheatmap R package (v1.0.12, clustering_method=“ward.D2”, scale=“row”). Differential Expression Analysis (DEA) was performed between the passage 3 and 6 for each group separately (KPT, T-LM, K-LM) with the limma R package (v.3.42.2 (38)). A gene was considerate as a Differentially Expressed Gene (DEG) if its adjusted p-value was ≤0.01 (Benjamini & Hochberg (BH) method (CIT4)). The results are summarized in the following table.

| | KPT | T-LM | K-LM |
|---|---|---|---|
| Up-regulated DEG | 454 | 714 | 223 |
| Down-regulated DEG | 210 | 145 | 92 |
| Total DEG | 664 | 859 | 315 |

Then, enrichment analysis was done for DEG sets of each group separately. To perform enrichment analyses we used hypergeometric test (enricher function of clusterProfiler R package (39); v3.10.1) with go_terms.mgi downloaded on Mouse Genome Database (MGD) at the Mouse Genome Informatics website (URL: http://www.informatics.jax.org (40) (04/2019). A GO term was considered significantly enriched if its adjusted p-value was ≤0.05 (BH method). Then, we computed a z-score value as an additional indicator of the direction of the dis-regulation of the GO term as: z-score=(up-down)/sqrt(count) where up/down are the number of assigned genes up-or down-regulated, respectively, in the GO term (41). Finally, we searched for commons GO terms between each possible combination of KPT, T-LM and K-LM groups. The top 5 GO terms were selected for each group and combined group based on the gene counts and the z-score.

### Biomarker Strategy

Using the transcriptomics data obtained from our cell lines, we generated a list of highly differentially and progressively expressed genes. This list corresponded to the intersection of the gene sets selected as described in the steps (i) and (ii).

### (i) Highly Differentially Expressed Genes Between P0 and P6.

To select genes with the highest differential expression between the parental cell line and the P6 passage, we used a z-score approach. First, we computed the log fold change (logFC) for each gene, and then the mean and standard deviation of all the logFC, obtaining at the end a z-score for each gene. A gene was considered as highly differentially expressed if its absolute z-score value was ≥2.58 (corresponding to a p-value of 0.01) and if its absolute logFC was ≥2.

(ii) Genes with Progressive Expression Patterns.

We captured genes with a progressive expression pattern following the same direction during all passages with the limma R package. For this, we identified DEG (adjusted p-value≤0.01) for each following comparison: P0-P6, P0-P2, P2-P3, P3-P4, P4-P5 and P5-P6. A gene was considered as progressive if it was differentially expressed for the P0-P6 comparison and if the direction of its differential expression was the same through all other comparisons. Stable states with no significant difference between P0-P2, P2-P3, P3-P4, P4-P5 and P5-P6 comparisons were allowed. To ensure that the genes were specific to each generated cell line, we then selected only progressive genes through all passages and late passages, by keeping progressive genes differentially expressed in P0-P6, P0-P6 and P4-P5, and P0-P6 and P5-P6. Next, from our selected highly differentially and progressively expressed gene list, we converted Mus Musculus gene names to Human gene names using the conversion table from Biomart website (https://www.ensembl.org (42)). Subsequently, we generated potentially clinically relevant gene signatures using the TCGA-KIRC cohort, as described in the following steps (iii) and (iv).

(iii) Selection of Prognostic Human Genes.

For each gene, the KIRC cohort was segregated in 3 groups based on the expression. Then, we fitted a Cox proportional hazard regression model based on overall survival (OS) and disease-free survival (DFS) time. The cox proportional and log rank hazard ratio (HR) values were computed according with the differential expression (up or down) of the gene identified in the previous steps. A gene was selected if its HR was ≥2 and if the adjusted p-value (BH method) of its log-rank test was ≤0.01 for OS or DFS.

### (iv) Aggregation of Relevant Genes as Signatures.

To measure the clinical relevance of the resulting signatures, we used the SigCheck R package (v2.18, (43)). To separate samples into groups, we computed a score for each sample which corresponded to the mean value over all the expression values in the signature (scoreMethod=“High” in the sigCheck function). Patients were then ranked by their scores and split in 3 groups (high, medium, low) to perform a log rank test and compute associated HR. Signatures were tested for OS of all patients and DFS of only non-metastatic MO patients.

### (v) Validation of the Signatures.

We validated the significance of each signature after adjustment for clinical variables (Fuhrman grade and TNM stage) with a multivariate Cox regression analysis (ggforest function). To show that the signatures were significantly more associated with OS and DFS outcomes than random predictors, we compared the performance of each signature with 1000 signatures composed of the same number of randomly-selected genes (sigCheckRandom function).

### TCGA KIRC Cohort

TCGA Kidney Renal Clear Cell Carcinoma (KIRC) HiSeqV2 data were downloaded from XenaBrowser (44). We chose log 2(x+1) transformed RSEM normalized count (version 2017-10-13) as recommended on its web site (https://xenabrowser.net/; page: «dataset:gene expression RNAseq-IlluminaHiSeq percentile»). We removed genes whose expression had null values in more than 2 third of all samples (2360 genes). Complementary associated clinical data were downloaded from cbioportal (www.cbioportal.org/, (45)). Only primary tumor samples were used remaining to 533 patients whose 352 had a MO status.

### Methylomics Data Generation and Analysis

Whole Genome Bisulfite Sequencing (WGBS) was performed for three replicates of P6 groups for KPT, T-LM and K-LM cell lines, and one for the parental P0 cell line at the GeT-PlaGe core facility (INRA Toulouse). WGBS libraries have been prepared according to Biooscientific's protocol using the Biooscientific NEXTflex™ Bisulfite Library Prep Kit for Illumina Sequencing. Briefly, DNA was fragmented by sonication, size selection was performed using Agencourt AMPure beads XP and adaptators were ligated for sequencing. Then, bisulfite treatment was performed for 2.5 hours using the EZ DNA Methylation-Gold™ Kit from Zymo Research, and 12 cycles of PCR were performed. Library quality was assessed using an Advanced Analytical Fragment Analyser, and libraries were quantified by qPCR using the Kapa Library Quantification Kit. WGBS experiments have been performed on an Illumina HiSeq3000 using a paired end read length of 2×150pb with the Illumina HiSeq3000 Reagent Kits. To determine conversion efficiency, fastq files were trimmed for adapters and low quality bases with Trim Galore (v0.4.4, calling cutadapt 1.3 then mapped to the pUC19 reference genome (pUC19.fa) with Bismark (v0.13.0 (46). Samtools (v0.1.19-44428cd (47) was used to duplicated reads. Then methylation calling was performed with remove Bismark_methylation_extractor. As methylated and non-methylated cytosine positions are known on the pUC19 reference genome, over and under-conversion could be assessed. Filtered fasq file were generated by CASAVA 2.17. Fastq files were aligned with Bismark (v0.17.1_dev) against the GRCm38.p5 Mus musculus genome (download from http://www.ensembl.org/, release 89) with following parameters-N 0 and -maxins 800. Bismark use Bowtie 2 (v2.3.4.3) and samtools (v1.9). Incomplete bisulfite conversion filtering was done on Bismark BAM files in order to remove reads that exceed a certain threshold of methylated calls in non-CG context. Then, de-duplication was applied (deduplicate_bismark) followed by the extraction of methylated positions (bismark_methylation_extractor). We then considered CG positions with at least 10 reads of coverage resulting in 106303 CGs on chromosomes 1-19 and X. PCA was performed on methylation frequencies of all CGs for P0 and P6 samples (function prcomp of stats R package (v3.6.2) with the parameter center=T).

### Human Patient Samples.

Patient samples (tumor tissue and plasma) from the UroCCR cohort were used with associated clinical data (clinicaltrial.gov, NCT03293563). Eligible patients for SUVEGIL and TORAVA trials were at least 18 years of age and had metastatic ccRCC histologically confirmed, with the presence of measurable disease according to Response Evaluation Criteria in Solid Tumors v1.1. For SUVEGIL and TORAVA cohorts, patients did not received previous systemic therapy for RCC, and were eligible for sunitinib or bevacizumab treatment in the first-line setting. Patients were ineligible if they had symptomatic or uncontrolled brain metastases, an estimated lifetime less than 3 months, uncontrolled hypertension or clinically significant cardiovascular events (heart failure, prolongation of the QT interval), history of other primary cancer. All patients gave written informed consent. Tumors were assessed at baseline and then every 12 weeks by thoracic, abdominal, pelvic and bone CT scans. Brain CT scans were performed in case of symptoms. This cohort includes patients from the SUVEGIL (24 patients) and TORAVA (35 patients) trials. The SUVEGIL trial (clinicaltrial.gov, NCT00943839) was a multi-center prospective single-arm study. The goal of the trial is to determine whether a link exists between the effectiveness of therapy with sunitinib malate and development of blood biomarkers in patients with kidney cancer. Patients received oral sunitinib (50 mg per day) once daily for 4 weeks (on days 1 to 28), followed by 2 weeks without treatment. Courses repeat every 6 weeks in the absence of disease progression or unacceptable toxicity. The TORAVA trial (clinicaltrial.gov, NCT00619268) was a randomized prospective study. Patient characteristics and results have been previously described (40). Briefly, patients aged 18 years or older with untreated metastatic ccRCC were randomly assigned (2: 1: 1) to receive the combination of bevacizumab (10 mg kg-1 iv every 2 weeks) and temsirolimus (25 mg iv weekly) IFN-α (9 mIU i.v. three times per week), or one of the standard treatments: sunitinib (50 mg per day orally for 4 weeks followed by 2 weeks off) (48). These studies were approved by the ethic committee at each participating center and run in agreement with the International Conference on Harmonization of Good Clinical Practice Guideline. Blood samples were collected during the inclusion visit (baseline).

Validation of Signatures in a Cohort Treated with Immunotherapy

The relevance of the KPT, K-LM and T-LM signatures were also tested in two cohorts treated with either everolimus (mTOR inhibitor, 130 patients) or nivolumab (anti-PD-1, 181 patients) immunotherapies (9). We used SigCheck R package (v2.22, (43) with sigCheck function (scoreMethod=“High”). Signatures were tested for OS and Progression-Free Survival (PFS). To show that the signatures were significantly more associated with OS and PFS outcomes than random predictors, we compared the performance of each signature with 1000 signatures composed of the same number of randomly-selected genes (sigCheckRandom function).

### Statistical Analyses

Mann-Whitney U test and unpaired two-tail Student t-test were used for in vivo and in vitro, respectively, experiments. p-values <0.05 were considered statistically significant (* p<0.05; ** p<0.01; *** p<0.001). Statistical analyses were performed using either R studio (R v3.6.2 (49), R studio v1.2.5033 (50)) or GraphPad Prism (version 6.00 for Windows, La Jolla California USA, www.graphpad.com). Survival analysis were performed using survival ((51), v3.2-7) survminer (v0.4.8). Analysis of transcriptomics, methylomics, enrichment and signature computations were performed using R.

### Results

### Generation of Enhanced Aggressiveness GFP-RENCA Cell Lines Using a Syngeneic Mouse Model of RCC

To identify the molecular mechanisms responsible for the development of primary and metastatic tumors in RCC, we generated mouse renal carcinoma RENCA cell lines of progressively enhanced aggressiveness and metastatic potential, and analyzed the transcriptomic profiles. To this aim, we firstly generated RENCA cells stably expressing GFP, for cancer cells identification, and serially passaged the generated GFP-RENCA cells in female BALB/c immunocompetent mice, based on the seminal work of Fidler (7). For the dissection and study of each step involved in tumor progression, we used three different GFP-RENCA injection-explant modalities, coupled with RNA extraction from the generated cell lines for gene expression analysis. The three implantation/injection modalities are described as follows: (i) Orthotopic implantation under the renal capsule; cancer cells were then explanted from formed tumors, and purified for subsequent re-implantation into a kidney of another mouse. (ii) Intravenous injection into the tail vein, in the absence of a primary tumor in the kidney; here, cancer cells were explanted from the metastases formed in the lungs. (iii) Orthotopic implantation under the renal capsule and GFP-RENCA collection from metastatic sites in the lungs; subsequently, cancer cells were re-implanted under the kidney capsule for the following passage(s). By this experimental strategy, we generated 67 different cell lines that can be grouped in three main categories, each one describing the different aspects of cancer progression, from primary tumor growth to metastasis formation: 1-Kidney Primary Tumor (KPT) cell lines, which were expected to reveal mechanisms relevant to primary tumor growth and invasion; 2-Tail-to-Lung Metastases (T-LM) cell lines, which described the key aspects of metastasis formation (i.e. survival in the bloodstream, evasion of immune response, colonization and growth in distant secondary organ, such as lungs); 3-Kidney-to-Lung metastases (K-LM) cell lines, that recapitulated the whole process of tumor progression, from primary tumor growth to metastasis formation. To note, each cell line was sequentially passaged in vivo for 6 cycles, using multiple mice per injection mode and per passage. To purify GFP-RENCA cells from primary tumors and lung metastases, collagenase digested tissues were maintained in culture for more than 10-15 days (see Methods section). This window of time allowed us to have a cancer cells purity similar to parental in vitro cultured GFP-RENCA cell line (data not shown) for the subsequent implantation/injection.

### Late Passage P6 Cell Lines Showed Enhanced Aggressiveness and Metastatic Potentials Compared to Earlier Passage P1 Cells

During the generation of the cell lines, we observed a passage-dependent reduction in mice survival time (from 26 to 15 days), suggesting that the cells became increasingly aggressive after each in vivo implantation/extraction cycle (data not shown). In fact, we observed that, after 15 days from orthotopic implantation, P6 KPT cells formed larger primary tumors compared to GFP-RENCA that underwent to only one cycle of injection (i.e. P1 cells), while P6 K-LM cell lines generated tumors that were of comparable weight with the ones formed by P1 cells (data not shown). Such a difference in tumor growth could be explained by an increase in the proliferation rate of P6 KPT cells, as shown by immunofluorescence analysis of the proliferative marker Ki67 in these samples (data not shown). Concomitantly with primary tumor growth, we also compared the metastatic potential of P6 cell lines to PI cells. In particular, we observed that P6 cells enhanced the formation of lung metastases compared to P1 cells, after 15 days from either orthotopic implantation or tail vein injection (data not shown). Furthermore, P6 cells changed their mode of growth in 2D-culture. In fact, RENCA cells normally form compact colonies. However, P6 cells were less adherent to each other, compared to parental non-implanted P0 cells, and unable to grow in clusters (data not shown). Such a phenotype, in addition to increased in vivo metastatic potential, suggested that P6 cells acquired an enhanced migratory ability, as further demonstrated by a Boyden chamber assay (data not shown). Furthermore, gene expression analysis of P6 of the 3 different groups revealed changes reminiscent for an EMT phenotype (data not shown).

### Global Functional Genomic Analysis of Amplified Mouse Cell Lines

In order to determine whether the phenotypic differences are due to genomic or epigenetic alterations, we first performed low-coverage whole-genome sequencing on P0 and P6 cells lines to assess whether copy number variability could possibly underlie the change in phenotype (data not shown). We failed, however, to observe significant differences in copy numbers between parental and passaged samples, both at the level of the number of breakpoints detected (45 for parental versus 41.75+6.32 for passaged lines) and the percentage of the genome with a copy number different than 2 (19% for parental versus 18%+1% for passaged lines) (data not shown). As the differences cannot be explained by genomic alterations we focused on transcriptomic and methylome analysis. We performed RNA extraction of GFP-RENCA cells isolated after each passage for transcriptomic analysis. We used Principal Component Analysis (PCA) to summarize the information contained in our data sets for cell passages P3, P5 and P6. This analysis revealed that, after each passage, clustering became more evident and cluster segregation specific for the respective implantation/injection mode. Thus, at latest passage P6, KPT, T-LM and K-LM cell lines clustered into three distinct groups (data not shown). We next selected all the genes that were major contributors to the Principal Component 1 (PC1) and 2 (PC2) and performed a heatmap of the transcriptomic profiles. The PI cell lines were excluded from the analysis because of insufficient number of replicates. This analysis revealed a gradual change in gene expression along with cell passages (data not shown). The PCA for the methylome data obtained by full methylome sequencing of P0 and P6 cells, showed similar clustering in 4 groups corresponding to KPT, T-LM, K-LM and parental P0 cell lines (data not shown). Gene Ontology (GO) enrichment analysis between P3 and P6 cell lines (see Materials and Methods for details) showed several highly enriched categories for each group (data not shown). These include processes that are in common for all three groups, for 2 groups or specific of each group. Common processes such collagen-containing ECM, cell adhesion, or extracellular matrix organization are required for cancer cells to evolve during the different steps of tumor progression. Others were more specific for one or two group(s) only, and are in favor of role in either primary tumor growth, metastatic spread or survival in the blood stream during the dissemination process or a combination of two of these processes. These include for instance categories such as regulation of cell population proliferation, positive regulation of cell migration, actin-binding, integrin binding, cytokine activity, TGF-b binding and immune system processes. This further indicates that the three groups of cell lines evolved differently up to the passage P6 by acquiring different regulatory modules. Inactive mutation of the VHL gene is a hallmark of renal carcinoma cells which is detectable in the majority of RCC patients. Despite the fact that our model of RCC was generated using a wtVHL cell line (i.e. RENCA), we observed that T-LM and K-LM late passage P6 cell lines displayed main characteristics of mutVHL RCC cells (i.e. enhanced metastatic potential and EMT). In a previous study, a set of upregulated gene with clinical impact has been identified in VHL-knockout RENCA cells (8). To assess the degree of VHL cascade activation in our cell lines, we compared the expression of this VHL knockout related genes to our transcriptomic dataset. The heatmap of T-LM and K-LM late passages cells (including P4-P5-P6) revealed clustering and increased expression of the VHL knockout related genes when compared to early passaged cells (data not shown). In particularly, these included four HIFla target genes (i.e. P0STN, TNFSF13B, PPEF1 and SAMSN1) that were significantly up-regulated in VHL-KO RENCA, and are of poorly prognosis for RCC patients (data not shown). However, KPT cell lines did not show evident clustering with the VHL-KO RENCA gene signature.

### Clinically Relevant Signatures and Biomarker Discovery

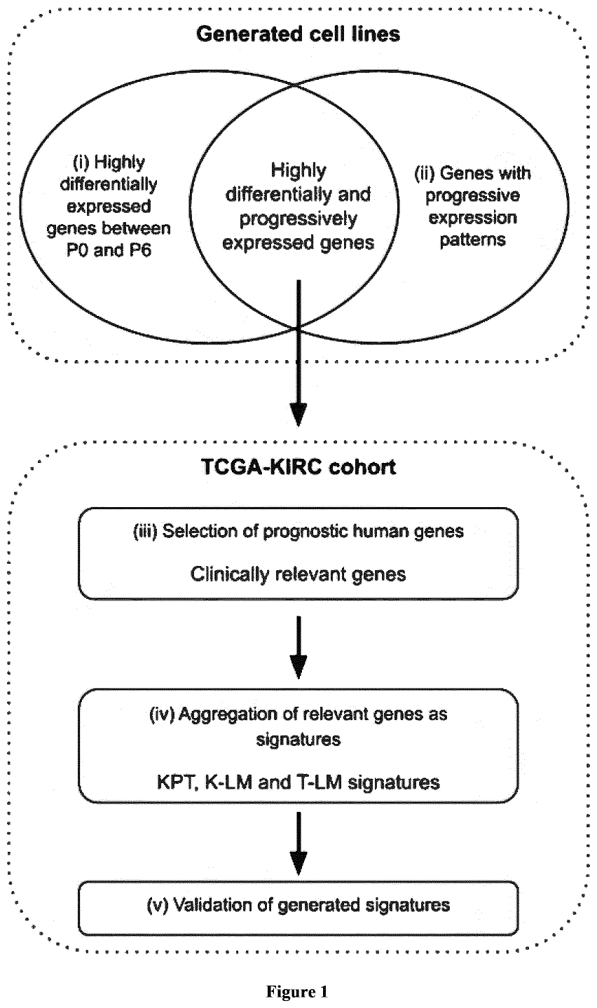

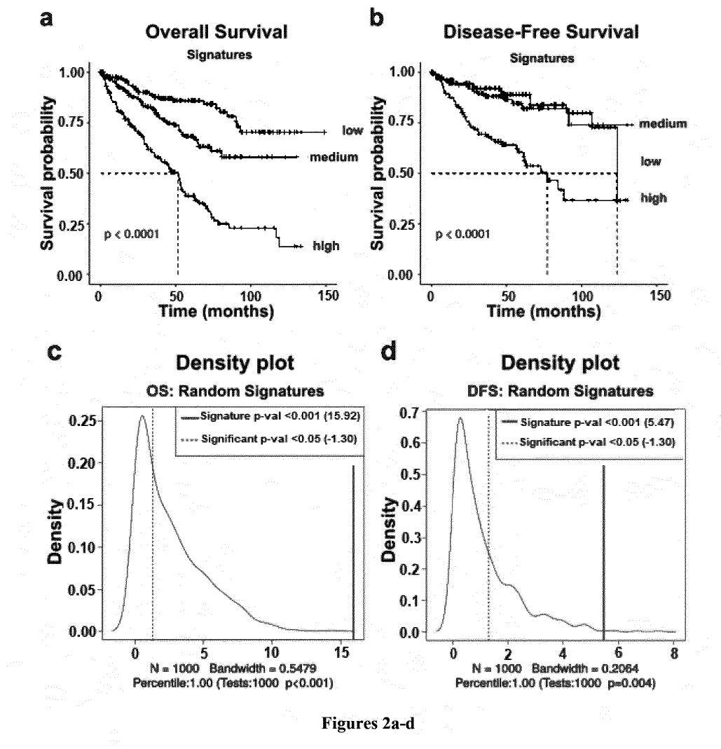

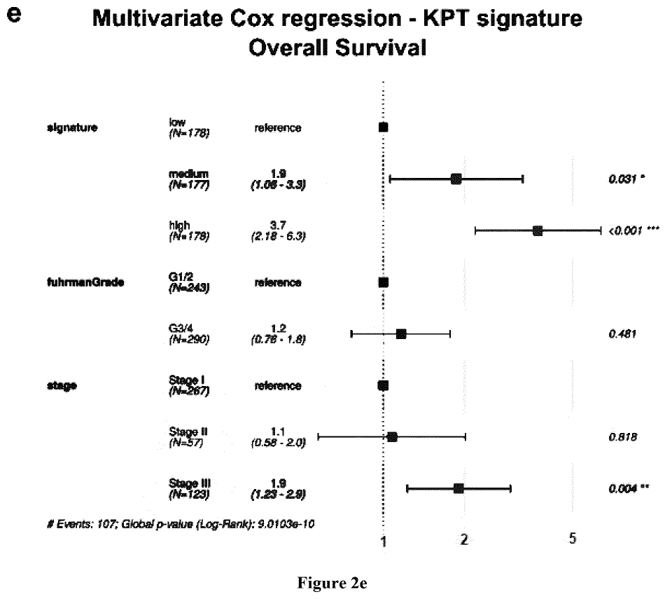

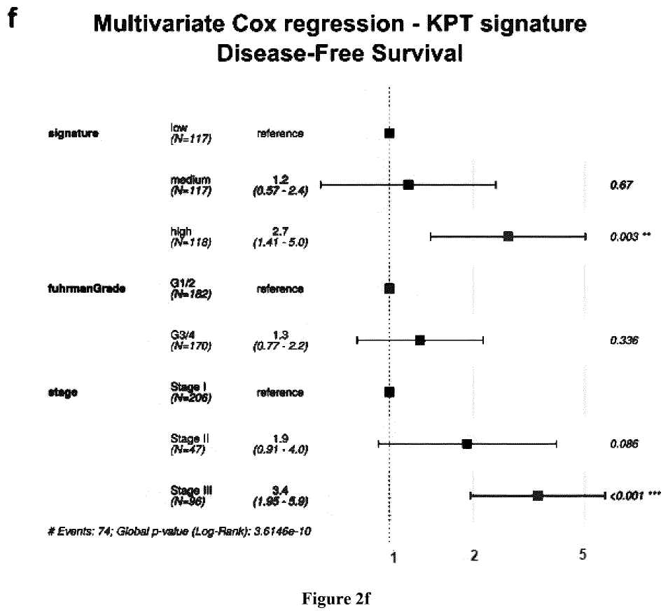

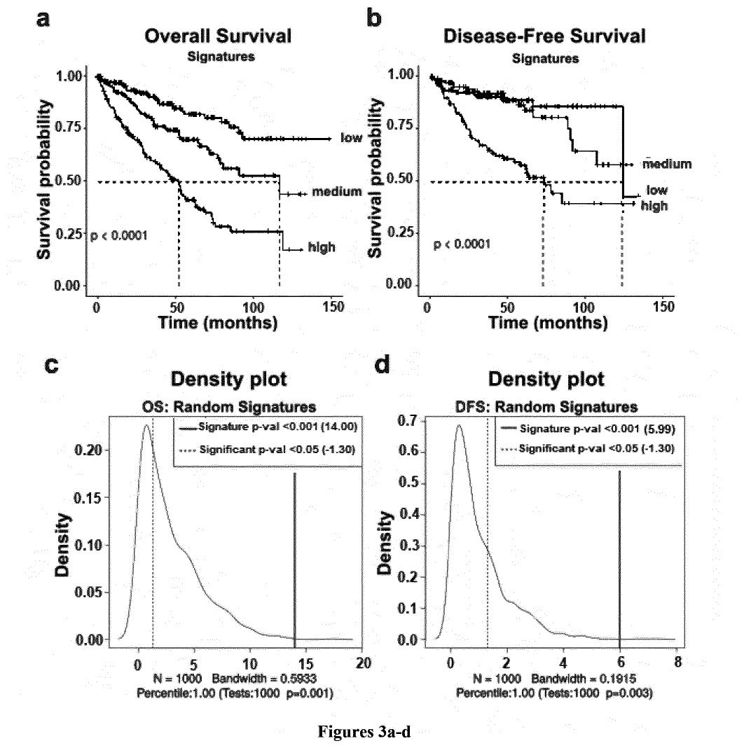

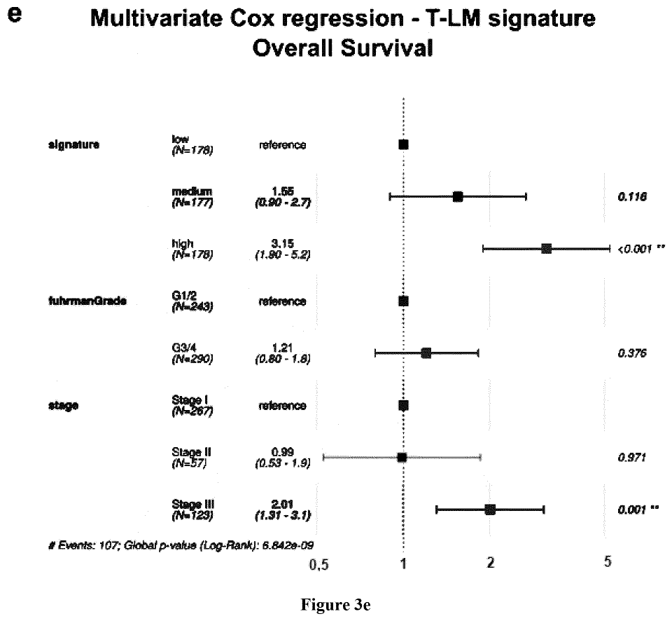

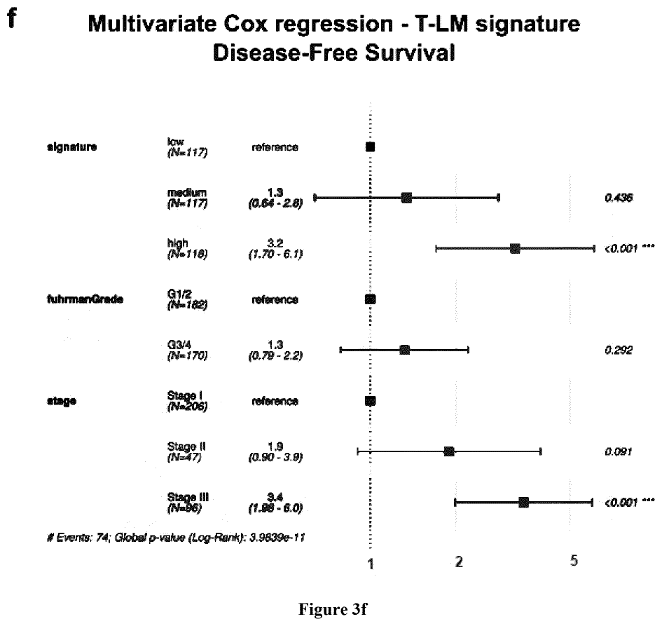

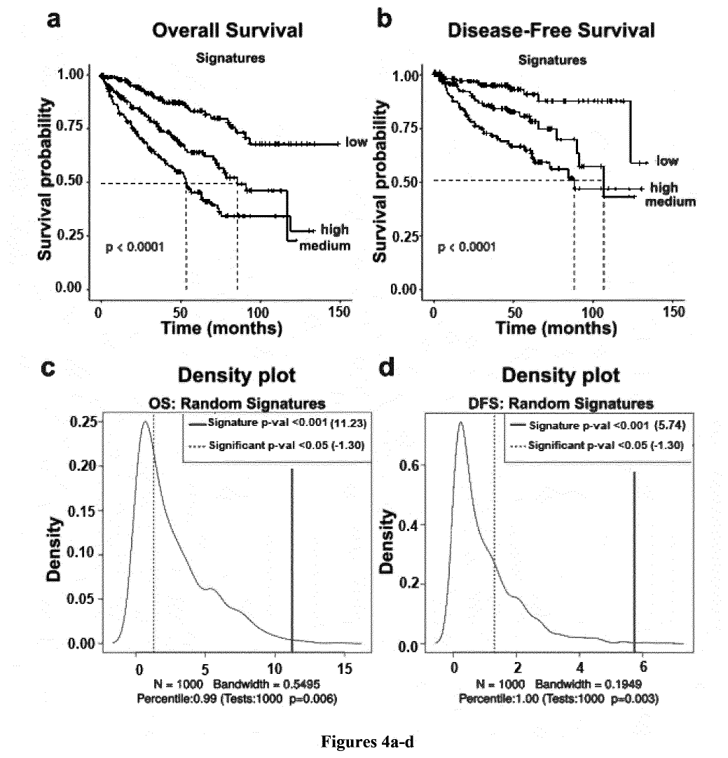

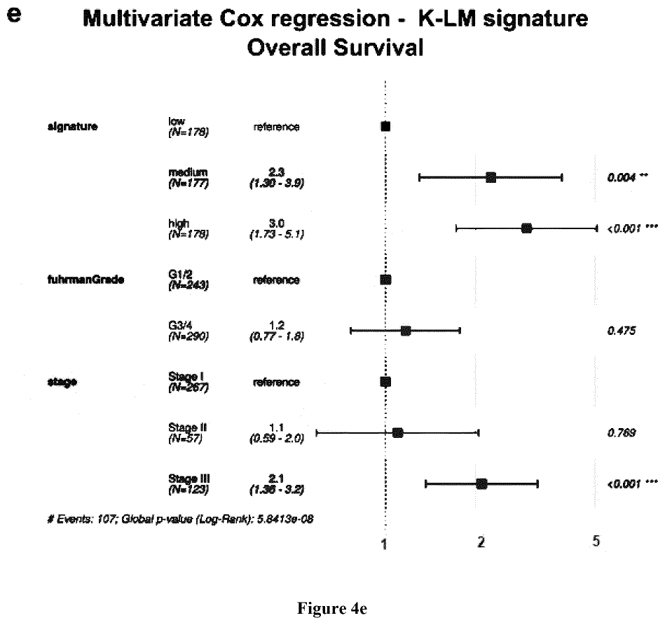

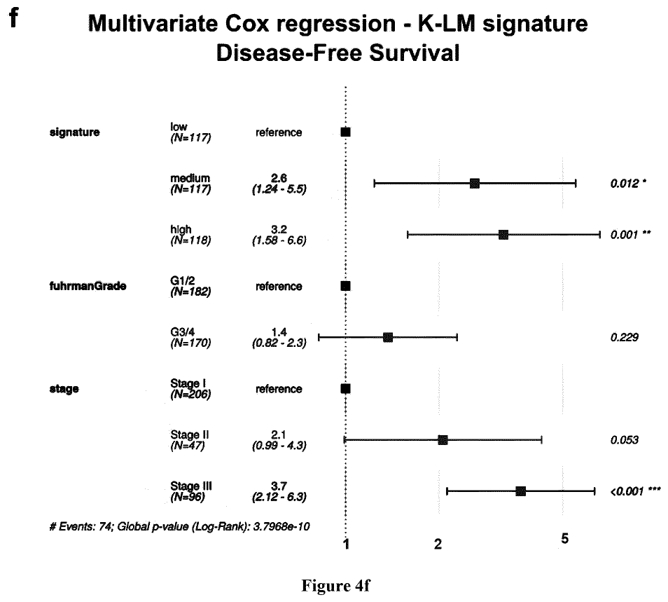

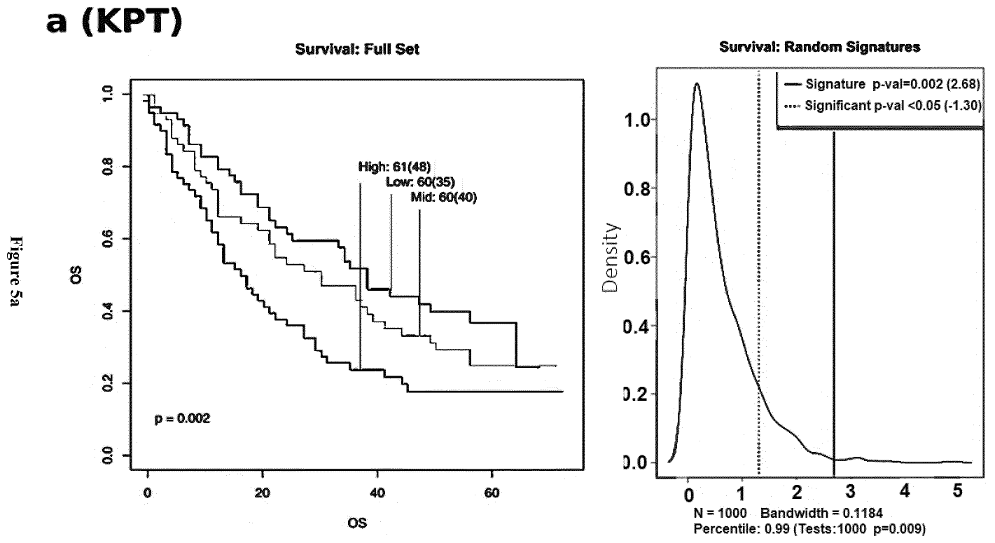

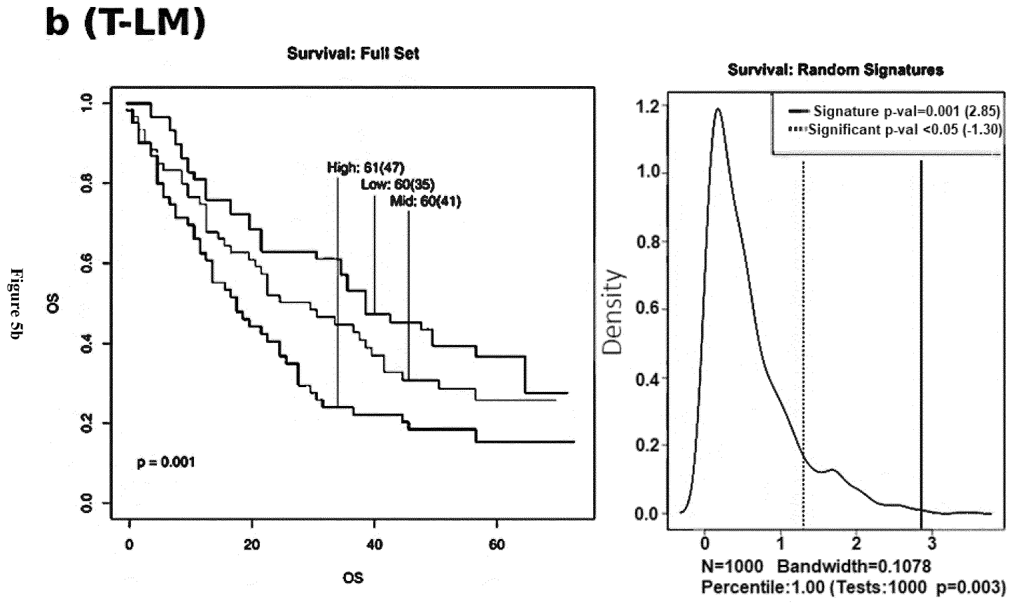

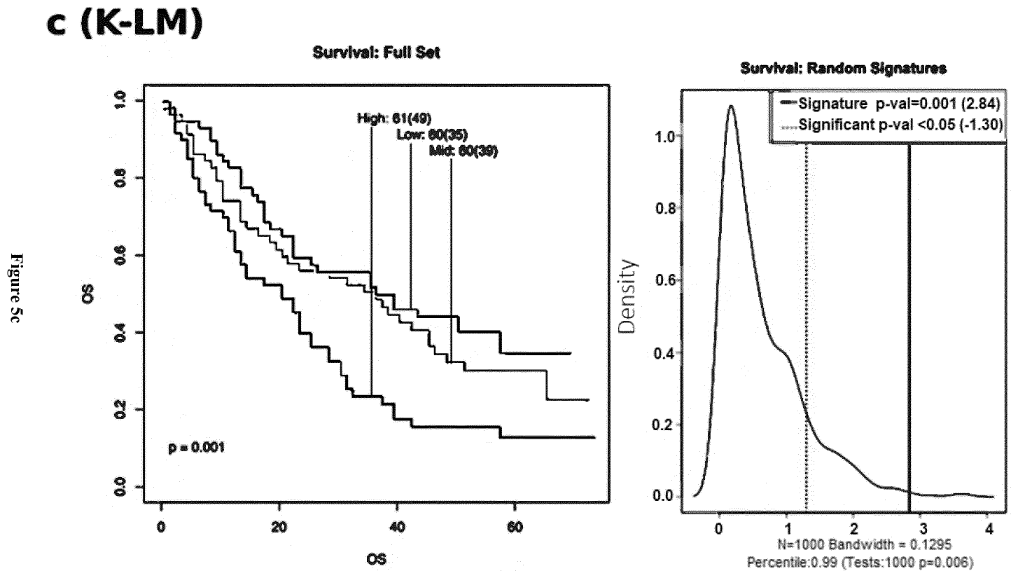

We next investigated whether transcriptional signatures derived from the analysis of differentially expressed genes in the KPT, T-LM and K-LM-groups could predict clinical outcome of patients. To this aim we used the Clear Cell Renal Cell Carcinoma dataset (KIRC) from The Cancer Genome Atlas (TCGA). The general strategy is outlined in FIG. 1. To select biomarkers from our mouse model, we compared genes that changed their expression between the parental P0 and P6 cell line and exhibited a progressive expression pattern (KPT, T-LM, K-LM). To determine the list of clinically relevant genes, we next investigated the predictive value of the 3 gene lists using TCGA (KIRC cohort). For each gene, we fitted a Cox-proportional hazard regression model based on overall survival (OS) or disease-free survival (DFS). A gene was conserved if its false-discovery rate (FDR) adjusted p-value of its log-rank test was lower than 0.01 and if the hazard ratio (HR) was consistent with the differential expression. To determine the specific signatures, we combined genes from the clinically relevant biomarker gene list. In order to validate our signature, we computed an empirical p-value by testing our signatures against 1000 random signatures of equivalent size. Furthermore, we performed multivariate Cox regression analysis of our signatures. The results for the 3 subgroups are depicted including the OS and DFS for MO patients (Table 1, FIGS. 2a,2b,2c,2d,2e,2f, 3a,3b,3c,3d,3e,3f and 4a,4b,4c,4d,4e,4f). After adjusting for clinical variables (TNM stage and Fuhrman grade), the K-LM signature had the best hazard ratio and remained an independent prognostic factor for predicting both OS and DFS. We next compared our signature in the data set reporting treatment of RCC by targeted therapy which included immunotherapy (9). All three signatures (KPT, K-LM, T-LM) were predictive for OS when assessed in the cohort treated with immunotherapy (FIG. 5a,b,c). The identification of signatures predictive of patient outcome also validates our experimental approach and shows that the strategy of generation of increasingly specialized mouse cell lines revealed novel genes and signatures with relevance to human RCC.

### DISCUSSION

The incidence and prevalence of RCC are rising, and mortality rates have barely improved over the last 20 years (2). This is in stark contrast with markedly improving survival rates in many other cancers and highlights RCC as one of the cancers in which current therapeutic approaches have failed to make the advances hoped for. Different drugs are available for RCC treatment, aiming to counteract its high angiogenic and immunogenic environment (15). However, the limitations associated with these drugs include treatment resistance and, thus, leading to failure of RCC eradication. The pathophysiology of RCC is still far from understood, and clinical treatment of RCC is hampered by a lack of validated molecular biomarkers. The elucidation of key mechanisms in RCC progression and the identification of novel biomarkers of RCC can open up novel therapeutic strategies targeting different aspects of RCC biology, including chemoresistance onset.

In this context, we have generated a mouse model designed to dissect different stages of RCC progression using the RENCA model. Although the usefulness of the RENCA model has been widely documented by many preclinical studies for RCC and led to meaningful results (16-19), these cells could represent a weak point of our model, as they bear wild-type VHL gene status. In fact, the majority of human RCC tumors are VHL-inactivated, questioning the clinical relevance of our model. In order to overcome this problem, we compared the transcriptomic profiles of our generated cell lines with RNA-seq analysis of VHL-KO RENCA cells, generated in a previous study by Schokrpur et al (8). In this study, authors provided a list of 10 up-regulated genes upon VHL deletion. We therefore analyzed their expression in our cell lines and observed that late passages K-LM and T-LM metastatic groups also have up-regulation of these genes, compared to earlier passages and parental P0 cell lines. These data suggest that K-LM or T-LM late passage cell lines can be helpful to describe the molecular changes occurring also in VHL-inactivated cancer cells.

To decipher the different steps of renal tumor development and dissemination, we produced mouse renal cancer RENCA cells of progressively enhanced aggressiveness and specialization. Using multiple implantation strategies, we analyzed different aspects of primary tumor growth and metastasis, and determined their transcriptomic profiles. This approach allowed us to monitor the expression changes of genes involved in the mechanisms underlying RCC development and progression, from primary tumor growth to metastasis formation. In fact, we generated three distinct lists of differentially and progressively expressed genes, based on the implantation-extraction modality that we used. These genes were then selected based on their prognostic values in TCGA-KIRC cohort, aggregated as signatures and finally validated.

Although serial tumor cells passaging in mice is an established technique to generate more aggressive cell lines, we are the first analyzing the transcriptional changes of the different cell lines. RNA expression and methylation analysis demonstrated distinct clustering for the three different injection modes. DNA sequencing did not show clonal variations based on chromosomal variability, which indicates that the phenotypic changes were epigenetically regulated. Transcriptomic analysis led to the identification of specific gene signatures for each injection cycle which were predictors of OS, DFS and PFS in ccRCC as based on the TCGA-KIRC cohort and on our two ccRCC cohorts. Importantly, the signatures, especially the K-LM signature, were stronger predictors than current predictors in clinical use such as Fuhrman grade or clinical stage. Our signature is different from the signature previously published such as ccrcc1-4 signatures (27), 16-gene assay (28) and ClearCode34 (29). Ricketts et al. (29) reported a comprehensive molecular characterization using the TCGA database where they compared the three types of RCC (ccRCC, papillary RCC and chromophobe RCC). For ccRCC, the signatures were related to increased ribose metabolism pathway and to Th2 immune profile. However, our study is very different because starting from an animal model, we specifically focused our comparative translational analysis on the ccRCC subtype and, thus, the results cannot be compared, albeit their study also revealed immunology-related gene expression. Furthermore, a recent study reported tracking of ccRCC evolution at the genomic level and demonstrated that metastatic competence was afforded by chromosomal complexity with loss of 9p as a selective event for metastasis and patient survival (30). However, our study did not reveal chromosomal alterations and, thus, we specifically focused on modifications in gene expression. Our signatures were also found to be predictive when analyzed in an immunotherapy data set which could of use in stratifying patients undergoing targeted therapy.

All in all, we described herein a series of systematic studies based on a model using serial implantations with the following strengths: (i) the use of three different modalities of tumor cell implantation-extraction, for the dissection of molecular mechanisms occurring in each step of tumor progression; (ii) the use of a syngeneic mouse model, which permitted to work with an intact immune system; (iii) the three different cell groups yielded very distinct transcriptomic and methylome clustering and signatures, which led to meaningful results for the clinic as well as to a computational model for predicting tumor relapse.

### Tables:

TABLE 1
KPT, K-LM and T-LM signatures.

| | | HR-logRank | | | empirical |
|---|---|---|---|---|---|
| Signature | HR (95% CI) | (95% CI) | p value - 2 arms | p value - 3 arms | p value |
| | OS | | | | |
| KPT | 5.081 (3.313- | 4.968 (3.495- | 1.201547672015e−16 | 1.17359079713023e−17 | 0 |
| | 7.791) | 7.06) | | | |
| K-LM | 4.734 (3.086- | 4.576 (3.215- | 1.00189519999304e−14 | 1.15350359003188e−15 | 0 |
| | 7.262) | 6.512) | | | |
| T-LM | 4.102 (2.666- | 4.002 (2.786- | 5.91218514487598e−12 | 3.93707813897723e−11 | 0 |
| | 6.313) | 5.749) | | | |
| | DFS | | | | |
| KPT | 3.694 (2.049- | 3.605 (2.168- | 3.39623504753034e−06 | 1.84927020926968e−07 | 0 |
| | 6.658) | 5.992) | | | |
| K-LM | 4.293 (2.314- | 4.096 (2.456- | 1.03266390103381e−06 | 1.94657564814182e−08 | 0 |
| | 7.964) | 6.833) | | | |
| T-LM | 4.752 (2.381- | 4.59 (2.664- | 1.82008612143742e−06 | 6.95908563678835e−06 | 0 |
| | 9.484) | 7.91) | | | |

### REFERENCES

Throughout this application, various references describe the state of the art to which this invention pertains. The disclosures of these references are hereby incorporated by reference into the present disclosure.
1. Molina A M, Motzer R J. Clinical Practice Guidelines for the Treatment of Metastatic Renal Cell Carcinoma: Today and Tomorrow. Oncologist [Internet]. Wiley; 2011 [cited 2021 May 20]; 16:45-50. Available from: https://pubmed.ncbi.nlm.nih.gov/21346039/2.
2. Ferlay J, Laversanne M, Ervik M, Lam F, Colombet M, Mery L, et al. Global Cancer Observatory: Cancer Tomorrow [Internet]. Int. Agency Res. Cancer. 2020 [cited 2021 May 20]. Available from: https://gco.iarc.fr/today
3 Acar Ö, Şanli Ö. Surgical Management of Local Recurrences of Renal Cell Carcinoma. Surg Res Pract. 2016;2016.
4. Ficarra V, Martignoni G, Lohse C, Novara G, Pea M, Cavalleri S, et al. External validation of the mayo clinic stage, size, grade and necrosis (SSIGN) score to predict cancer specific survival using a European series of conventional renal cell carcinoma. J Urol [Internet]. 2006; 175:1235-9. Available from: http://www.jurology.com/doi/10.1016/S0022-5347%2805%2900684-1
5. Heng DYC, Xie W, Regan M M, Harshman L C, Bjarnason G A, Vaishampayan UN, et al. Consortium prognostic model: a population-based study. Lancet Oncol. 2014; 14:141-8.
6. Gupta G P, Massagué J. Cancer Metastasis: Building a Framework [Internet]. Cell. Elsevier B.V.; 2006 [cited 2021 May 20]. page 679-95. Available from: https://pubmed.ncbi.nlm.nih.gov/17110329/7.
7. Fidler I J. Selection of successive tumour lines for metastasis. Nat New Biol [Internet]. 1973; 242:148-9. Available from: http://www.ncbi.nlm.nih.gov/pubmed/4512654
8. Schokrpur S, Hu J, Moughon D L, Liu P, Lin L C, Hermann K, et al. CRISPR-Mediated VHL Knockout Generates an Improved Model for Metastatic Renal Cell Carcinoma. Sci Rep [Internet]. Nature Publishing Group; 2016 [cited 2021 May 20];6. Available from: https://pubmed.ncbi.nlm.nih.gov/27358011/9
9. Braun D A, Hou Y, Bakouny Z, Ficial M, Sant′ Angelo M, Forman J, et al. Interplay of somatic alterations and immune infiltration modulates response to PD-1 blockade in advanced clear cell renal cell carcinoma. Nat Med [Internet]. Nature Research; 2020 [cited 2021 May 21];26:909-18. Available from: https://pubmed.ncbi.nlm.nih.gov/32472114/WO 2023/280790 42-PCT/EP2022/068483
10. Hansen M T, Forst B, Cremers N, Quagliata L, Ambartsumian N, Grum-Schwensen B, et al. A link between inflammation and metastasis: Serum amyloid Al and A3 induce metastasis, and are targets of metastasis-inducing S100A4. Oncogene [Internet]. Nature Publishing Group; 2015 [cited 2019 Nov. 25];34:424-35. Available from: http://www.nature.com/articles/onc2013568
11. Baratchart E, Benzekry S, Bikfalvi A, Colin T, Cooley L S, Pineau R, et al. Computational Modelling of Metastasis Development in Renal Cell Carcinoma. Komarova N L, editor. PLOS Comput Biol [Internet]. 2015 [cited 2018 Apr. 13]; 11:e1004626. Available from: http://dx.plos.org/10.1371/journal.pcbi.1004626
12. Benzekry S, Tracz A, Mastri M, Corbelli R, Barbolosi D, Ebos JML. Modeling spontaneous metastasis following surgery: An in vivo-in silico approach. Cancer Res [Internet]. American Association for Cancer Research Inc.; 2016 [cited 2021 May 20];76:535-47. Available from: https://pubmed.ncbi.nlm.nih.gov/26511632/13.
13. Nicolò C, Perier C, Prague M, Bellera C, MacGrogan G, Saut O, et al. Machine Learning and Mechanistic Modeling for Prediction of Metastatic Relapse in Early-Stage Breast Cancer. JCO Clin Cancer Informatics [Internet]. American Society of Clinical Oncology (ASCO); 2020 [cited 2021 May 20];4:259-74. Available from: https://pubmed.ncbi.nlm.nih.gov/32213092/14.
14. Álvarez-Arenas A, Souleyreau W, Bikfalvi A, Emanuelli A, Bernhard J C, Benzekry S. Identifiability analysis of a mechanistic model for the time to distant metastatic relapse and its application to renal cell carcinoma. Plos Comp Bio (in prep).
15. Kotecha R R, Motzer R J, Voss M H. Towards individualized therapy for metastatic renal cell carcinoma [Internet]. Nat. Rev. Clin. Oncol. Nature Publishing Group; 2019 [cited 2021 Jun. 4]. page 621-33. Available from: https://pubmed.ncbi.nlm.nih.gov/30992569/16. Hara T, Miyake H, Hinata N, Fujisawa M. Inhibition of Tumor Growth and Sensitization to Sunitinib by RNA Interference Targeting Programmed Death-ligand 1 in Mouse Renal Cell Carcinoma RenCa Model. Anticancer Res. 2019; 39:4737-42.
17. O'Shaughnessy M J, Murray K S, La Rosa S P, Budhu S, Merghoub T, Somma A, et al. Systemic antitumor immunity by PD-1/PD-L1 inhibition is potentiated by vascular-targeted photodynamic therapy of primary tumors. Clin Cancer Res [Internet]. American Association for Cancer Research Inc.; 2018 [cited 2020 Jun. 24];24:592-9. Available from: https://pubmed.ncbi.nlm.nih.gov/28954788/18.
18. Balan M, Mier Y Teran E, Waaga-Gasser A M, Gasser M, Choueiri T K, Freeman G, et al. Novel roles of c-met in the survival of renal cancer cells through the regulation of HO-1 and PD-L1 expression. J Biol Chem [Internet]. American Society for Biochemistry and Molecular Biology Inc.; 2015 [cited 2020 Jun. 24];290:8110-20. Available from: https://pubmed.ncbi.nlm.nih.gov/25645920/19.
19. Sobczuk P, Brodziak A, Khan M I, Chhabra S, Fiedorowicz M, Wełniak-Kamińska M, et al. Choosing The Right Animal Model for Renal Cancer Research. Transl Oncol [Internet]. 2020; 13:100745. Available from: https://linkinghub.elsevier.com/retrieve/pii/S1936523319306394
20. Hou W, Ji Z. Generation of autochthonous mouse models of clear cell renal cell carcinoma: mouse models of renal cell carcinoma. Exp Mol Med [Internet]. 2018 [cited 2019 Nov. 25];50:30. Available from: http://www.nature.com/articles/s12276-018-0059-4
21. Bailey S T, Smith A M, Kardos J, Wobker S E, Wilson H L, Krishnan B, et al. MYC activation cooperates with Vhl and Ink4a/Arf loss to induce clear cell renal cell carcinoma. Nat Commun [Internet]. 2017 [cited 2019 Nov. 25];8:15770. Available from: http://www.nature.com/articles/ncomms15770
22. Gu Y F, Cohn S, Christie A, Mckenzie T, Wolff N, Do QN, et al. Modeling renal cell carcinoma in mice: Bap1 and Pbrml inactivation drive tumor grade. Cancer Discov [Internet]. 2017 [cited 2019 Nov. 25];7:900-17. Available from: http://www.ncbi.nlm.nih.gov/pubmed/28473526
23. Di Martino S, De Luca G, Grassi L, Federici G, Alfonsi R, Signore M, et al. Renal cancer: New models and approach for personalizing therapy. J Exp Clin Cancer Res [Internet]. 2018; 37:217. Available from: https://jeccr.biomedcentral.com/articles/10.1186/s13046-018-0874-4
24. Teicher B A, Hillman G G. Experimental Animal Models for Renal Cell Carcinoma. Tumor Model Cancer Res [Internet]. Humana Press, Totowa, NJ; 2003 [cited 2019 Dec. 2];493-505. Available from: https://link.springer.com/chapter/10.1007/978-1-59259-100-8 26
25. Schueler J, Klingner K, Bug D, Zoeller C, Maier A, Dong M, et al. Patient derived renal cell carcinoma xenografts exhibit distinct sensitivity patterns in response to antiangiogenic therapy and constitute a suitable tool for biomarker development. Oncotarget [Internet]. Impact Journals, LLC; 2018 [cited 2019 Dec. 2];9:30946-61. Available from: http://www.ncbi.nlm.nih.gov/pubmed/30123419
26. Patel A, Cohen S, Moret R, Maresh G, Gobe G C, Li L. Patient-derived xenograft models to optimize kidney cancer therapies. Transl. Androl. Urol. 2019. page S156-65.
27. Verbiest A, Couchy G, Job S, Zucman-Rossi J, Caruana L, Lerut E, et al. Molecular Subtypes of Clear Cell Renal Cell Carcinoma Are Associated With Outcome During Pazopanib Therapy in the Metastatic Setting. Clin Genitourin Cancer [Internet]. 2018 [cited 2019 Dec. 20]; 16:e605-12. Available from: http://www.ncbi.nlm.nih.gov/pubmed/29239846
28. Rini B, Goddard A, Knezevic D, Maddala T, Zhou M, Aydin H, et al. A 16-gene assay to predict recurrence after surgery in localised renal cell carcinoma: Development and validation studies. Lancet Oncol [Internet]. Lancet Publishing Group; 2015 [cited 2020 Jun. 29]; 16:676-85. Available from: https://pubmed.ncbi.nlm.nih.gov/25979595/29.
29. Brooks S A, Brannon A R, Parker J S, Fisher J C, Sen O, Kattan MW, et al. ClearCode34: A prognostic risk predictor for localized clear cell renal cell carcinoma. Eur Urol [Internet]. Elsevier; 2014 [cited 2020 Jun. 29];66:77-84. Available from: /pmc/articles/PMC4058355/?report=abstract
30. Turajlic S, Xu H, Litchfield K, Rowan A, Chambers T, Lopez JI, et al. Tracking Cancer Evolution Reveals Constrained Routes to Metastases: TRACERx Renal. Cell [Internet]. 2018 [cited 2019 Dec. 16]; 173:581-594.e12. Available https://linkinghub.elsevier.com/retrieve/pii/S0092867418303891 from:
31. Wood S L, Rogers M, Cairns D A, Paul A, Thompson D, Vasudev NS, et al. Association of serum amyloid A protein and peptide fragments with prognosis in renal cancer. Br J Cancer [Internet]. 2010 [cited 2019 Nov. 27]; 103:101-11. Available from: http://www.ncbi.nlm.nih.gov/pubmed/20531413
32. Vermaat J S, van der Tweel I, Mehra N, Sleijfer S, Haanen J B, Roodhart J M, et al. Two-protein signature of novel serological markers apolipoprotein-A2 and serum amyloid alpha predicts prognosis in patients with metastatic renal cell cancer and improves the currently used prognostic survival models. Ann Oncol. 2009; 21:1472-81.
33. Vermaat J S, Gerritse F L, Van Der Veldt A A, Roessingh W M, Niers™, Oosting SF, et al. Validation of serum amyloid a as an independent biomarker for progression-free and overall survival in metastatic renal cell cancer patients. Eur Urol [Internet]. 2012 [cited 2019 Nov. 27];62:685-95. Available from: http://www.ncbi.nlm.nih.gov/pubmed/22285764
34. Kim S H, Lee M J, Hwang H K, Lee S H, Kim H, Paik YK, et al. Prognostic potential of the preoperative plasma complement factor B in resected pancreatic cancer: A pilot study. Cancer Biomarkers [Internet]. 2019 [cited 2019 Nov. 27];24:335-42. Available from: http://www.ncbi.nlm.nih.gov/pubmed/30829612
35. Li H, Durbin R. Fast and accurate short read alignment with Burrows-Wheeler transform. Bioinformatics [Internet]. 2009 [cited 2019 Nov. 28];25:1754-60. Available from: https://academic.oup.com/bioinformatics/article-lookup/doi/10.1093/bioinformatics/btp324
36. Van Loo P, Nordgard S H, Lingjærde OC, Russnes H G, Rye I H, Sun W, et al. Allele-specific copy number analysis of tumors. Proc Natl Acad Sci USA [Internet]. 2010 [cited 2019 November 28]; 107:16910-5. Available from: http://www.pnas.org/cgi/doi/10.1073/pnas. 1009843107
37. Carter S L, Cibulskis K, Helman E, Mckenna A, Shen H, Zack T, et al. Absolute quantification of somatic DNA alterations in human cancer. Nat Biotechnol [Internet]. 2012 [cited 2019 Nov. 28];30:413-21. Available from: http://www.nature.com/articles/nbt.2203
38. Ritchie M E, Phipson B, Wu D, Hu Y, Law C W, Shi W, et al. Limma powers differential expression analyses for RNA-sequencing and microarray studies. Nucleic Acids Res [Internet]. 2015;43:e47. Available from: http://www.ncbi.nlm.nih.gov/pubmed/25605792
39. Yu G, Wang L G, Han Y, He Q Y. ClusterProfiler: An R package for comparing biological themes among gene clusters. Omi A J Integr Biol. 2012; 16:284-7.
40. Bult C J, Blake J A, Smith C L, Kadin J A, Richardson J E, Anagnostopoulos A, et al. Mouse Genome Database (MGD) 2019. Nucleic Acids Res [Internet]. 2019;47:D801-6. Available from: https://academic.oup.com/nar/article/47/D1/D801/5165331
41. Walter W, Sánchez-Cabo F, Ricote M. GOplot: An R package for visually combining expression data with functional analysis. Bioinformatics. 2015; 31:2912-4.
42. Aken B L, Achuthan P, Akanni W, Amode M R, Bernsdorff F, Bhai J, et al. Ensembl 2017. Nucleic Acids Res [Internet]. Narnia; 2017 [cited 2019 Nov. 25];45:D635-42. Available from: https://academic.oup.com/nar/article-lookup/doi/10.1093/nar/gkw1104
43. Venet D, Dumont J E, Detours V. Most random gene expression signatures are significantly associated with breast cancer outcome. PLOS Comput Biol [Internet]. PLOS Comput Biol; 2011 [cited 2021 May 20]; 7. Available from: https://pubmed.ncbi.nlm.nih.gov/22028643/44.
44. Goldman M, Craft B, Hastie M, Repečka K, McDade F, Kamath A, et al. The UCSC Xena platform for public and private cancer genomics data visualization and interpretation. bioRxiv [Internet]. 2019;326470. Available from: https://doi.org/10.1101/326470
45. Gao J, Aksoy B A, Dogrusoz U, Dresdner G, Gross B, Sumer SO, et al. Integrative analysis of complex cancer genomics and clinical profiles using the cBioPortal. Sci Signal [Internet]. Sci Signal; 2013 [cited 2021 May 20];6. Available from: https://pubmed.ncbi.nlm.nih.gov/23550210/46.
46. Krueger F, Andrews S R. Bismark: A flexible aligner and methylation caller for Bisulfite-Seq applications. Bioinformatics. 2011; 27:1571-2.
47. Li H, Handsaker B, Wysoker A, Fennell T, Ruan J, Homer N, et al. The Sequence Alignment/Map format and SAMtools. Bioinformatics. 2009; 25:2078-9.
48. Négrier S, Gravis G, Pérol D, Chevreau C, Delva R, Bay JO, et al. Temsirolimus and bevacizumab, or sunitinib, or interferon alfa and bevacizumab for patients with advanced renal cell carcinoma (TORAVA): A randomised phase 2 trial. Lancet Oncol [Internet]. 2011 [cited 2019 Nov. 27]; 12:673-80. Available from: http://www.ncbi.nlm.nih.gov/pubmed/21664867
49. R Core Team. A Language and Environment for Statistical Computing [Internet]. R Found. Stat. Comput. 2018. Available from: http://www.r-project.org
50. RStudio Team. RStudio: Integrated Development for R. RStudio, PBC, Boston, MA [Internet]. 2020 [cited 2021 May 20]. Available from: https://support.rstudio.com
51. Therneau™, Grambsch P M. Modeling Survival Data: Extending the Cox Model [Internet]. New York, NY: Springer New York; 2000 [cited 2021 May 20]. Available from: http://link.springer.com/10.1007/978-1-4757-3294-8

1. A method for predicting the survival time of a patient suffering from a renal cell carcinoma (RCC) and treating the patient comprising determining the expression levels of biomarkers
i) ADAM8, ARHGAP33, BTG3, COL6A1, CYBA, DNAJC12, DYRK4, FKBP10, IL34, MTMR7, PADI3, PLAU, RCN3, and TPRG1,
ii) ARHGAP33, BTG3, C4orf48, CKAP4, CRABP2, CYP3A4, DNAJC12, DYRK4, EREG, GFPT2, HISTIHIE, KCTD17, KDELR3, MMP14, NCAMI, NME4, PIGZ, PLAU, PLOD2, RGS19, SERPINA3, TBX4, TMEM45A, and TPRG1, and/or
iii) BCL3, CFB, COL6A1, CYP3A4, IQSEC3, KCTD17, KRT19, LOXL2, LRG1, PCBP3, SAA1, SAA2, SERPINA3, SOCS3, UCK2, and WT1;
identifying a patient having expression levels that are higher than predetermined reference values as having a shorter survival time; and
administering to the patient having a shorter survival time a therapeutically effective amount of at least one of an anti-cancer chemotherapeutic agent and an anti-cancer immunotherapeutic agent.

2. (canceled)

3. (canceled)

4. The method of claim 1, wherein the sample is a tissue tumor sample.

5. The method of claim 1, wherein a score which is a composite of the expression levels of the different biomarkers is determined and compared to the predetermined reference values wherein a difference between said score and said predetermined reference values is indicative whether the patient will have a long or short survival time.

6. The method of claim 1, for further comprising a step of selecting a therapeutic regimen or determining if a therapeutic regimen is appropriate for the patient identified as having a poor prognosis.

7. The method of claim 1, further comprising, after the step of administering, conducting follow-up testing, identifying the patient as having a good prognosis, and administering to the patient a maintenance dose of one or both of an anti-cancer chemotherapeutic agent and/or an anti-cancer immunotherapeutic agent.

8. (canceled)

9. The method of claim 1, wherein the anti-cancer chemotherapeutic agent is an anti-VEGF agent.

10. The method of claim 9 wherein the anti-VEGF agent is sunitinib.

11. The method of claim 1, wherein the anti-cancer immunotherapeutic agent is an immune checkpoint inhibitor.

12. The method of claim 11 wherein the immune checkpoint inhibitor is an anti-PD-1 antibody.

13. The method of claim 12 wherein the anti-PD-1 antibody is nivolumab.
